(19) United States
(12) Patent Application Publication
(10) Pub. No.: US 2023/0089914 A1
(43) Pub. Date: Mar. 23, 2023

(54) COMPOSITIONS COMPRISING AXITINIB AND METHODS OF TREATING OCULAR DISORDERS

(71) Applicant: Clearside Biomedical, Inc., Alpharetta, GA (US)

(72) Inventors: Viral KANSARA, Alpharetta, GA (US); Thomas CIULLA, Alpharetta, GA (US); Colette HALL, Alpharetta, GA (US); Barry KAPIK, Alpharetta, GA (US)

(21) Appl. No.: 17/800,874

(22) PCT Filed: Feb. 19, 2021
(86) PCT No.: PCT/US21/18737

§ 371 Date: Aug. 18, 2022

Related U.S. Application Data
(60) Provisional application No. 62/978,540, filed on Feb. 19, 2020.

(51) Int. Cl. A61K 31/4439; A61K 9/00; A61K 38/17; A61K 39/395; A61K 31/7105; A61K 47/26; A61K 47/38; A61K 47/02; A61P 27/02

(52) U.S. Cl. CPC A61K 31/4439; A61K 9/0048; A61K 38/179; A61K 39/3955; A61K 31/7105; A61K 47/26; A61K 47/38; A61K 47/02; A61P 27/02

(57) ABSTRACT

This disclosure is generally related to ophthalmic therapies, and more particularly to methods and devices that allow for infusion of a fluid drug formulation into posterior ocular tissues for targeted, localized treatment, for example, for the treatment of wet AMD in human patients. The drug formulation may be injected into the suprachoroidal space (SCS) or subretinal space of an eye. The drug formulation may include a tyrosine kinase inhibitor, such as axitinib.

### CROSS-REFERENCE TO RELATED APPLICATIONS

This application claims priority to U.S. Provisional Application No. 62/978,540, filed Feb. 19, 2020; which is incorporated herein by reference in its entirety.

### BACKGROUND OF THE INVENTION

Neovascular age-related macular degeneration (nAMD), also known as “wet” AMD, is the leading cause of vision loss in the industrialized world. It is caused by abnormal blood vessel growth in the choriocapillaris, a layer of capillaries situated immediately below Bruch's membrane. Choroidal neovascularization leads to the leakage of blood, lipids, and serum into the retinal layers, which can result in permanent damage to light-sensitive retinal cells and irreversible central vision distortions.

Although not fully elucidated, much is known regarding the pathogenesis of nAMD. The vascular endothelial growth factor (VEGF) signaling pathway has been shown to be centrally involved in the 10 to 15% of AMD diagnoses classified as the neovascular type (nAMD). In this pathway, VEGF signaling ligands bind to different isoforms of VEGF receptors (VEGFR) to activate cellular processes that promote growth of new vasculature. Specifically, VEGF-A, which acts at VEGFR-1 and -2, has been shown to promote abnormal blood vessel growth and is therefore an optimal target for treatment. Currently, anti-VEGF-A drugs are the standard of care for the treatment of nAMD; however, an unmet need remains for significantly improving and maintaining visual acuity in patients

Choroidal neovascularization (CNV) is the creation of new blood vessels in the choroid layer of the eye. Choroidal neovascularization is a common cause of, and/or can be associated with, nAMD. CNV can occur rapidly in individuals with defects in Bruch's membrane, the innermost layer of the choroid. It is also associated with excessive amounts of VEGF. CNV can also occur frequently with the rare genetic disease pseudoxanthoma elasticum and rarely with the more common optic disc drusen. CNV has also been associated with extreme myopia or malignant myopic degeneration, where in choroidal neovascularization occurs primarily in the presence of cracks within the retinal macular tissue known as lacquer cracks. CNV can create a sudden deterioration of central vision, noticeable within a few weeks. Other symptoms which can occur include color disturbances, and metamorphopsia (distortions in which straight lines appears wavy). Hemorrhaging of the new blood vessels can accelerate the onset of symptoms of CNV.

Currently, even the most successful treatments of nAMD and CNV do not preclude reoccurrence, making multiple treatments likely. In addition, currently available treatments do not restore vision that has already been lost. Therefore, there is a need in the art for treatment breakthroughs, in order to maintain vision for a longer period of time without repeated laser use.

### SUMMARY OF THE INVENTION

Methods and compositions for the treatment of neovascular age-related macular degeneration (nAMD; also referred to herein as wet AMD), choroidal neovascularization (CNV), nAMD associated with CNV, retinopathy, diabetic retinopathy, diabetic macular edema (DME), and related diseases are provided. The compositions comprise one or more tyrosine kinase inhibitors and are delivered to the suprachoroidal space of the eye via a non-surgical means. In some embodiments the tyrosine kinase inhibitor has activity against vascular endothelial growth factor (VEGF) and/or platelet derived growth factor (PDGF). In some embodiments, the tyrosine kinase inhibitor is axitinib. In some embodiments, the present disclosure provides an axitinib formulation, CLS-AX.

In some embodiments, the present disclosure provides methods for treating nAMD, choroidal neovascularization (CNV), nAMD associated with CNV, and/or DME comprising administering a formulation comprising axitinib to the suprachoroidal space of an eye of a subject in need thereof. In some embodiments, the method comprises administering about 0.01 mg to about 3.0 mg of axitinib to the eye. In some embodiments, the method comprises administering about 0.01 mg to about 0.5 mg of axitinib to the eye. In some embodiments, the method comprises administering about 0.01 mg to about 0.3 mg of axitinib to the eye. In further embodiments, the method comprises administering about 0.03 to about 0.1 mg of axitinib to the eye. In some embodiments, the method comprises administering about 0.01, about 0.02, about 0.03, about 0.04, about 0.05, about 0.06, about 0.07, about 0.08, about 0.09, about 0.10, about 0.2, about 0.3, about 0.4, about 0.5, about 1.0, about 2.0, or about 3.0 mg of axitinib to the eye. In some embodiments, the method comprises administering about 0.03 mg of axitinib to the eye. In some embodiments, the method comprises administering about 0.06 mg of axitinib to the eye. In some embodiments, the method comprises administering about 0.1 mg of axitinib to the eye. In some embodiments, the method comprises administering about 0.3 mg of axitinib to the eye. In some embodiments, the axitinib is administered in a volume of about 100 μL. Thus, in some embodiments, the method comprises administering an axitinib formulation in a concentration of about 0.1 mg/mL, to about 1 mg/mL, or about 0.1 mg/mL, about 0.3 mg/mL, about 0.6 mg/mL, or about 1.0 mg/mL. In some embodiments, the method comprises administering the axitinib to the eye non-surgically. For example, in embodiments, the method comprises administering the axitinib to the eye non-surgical surpachoroidal injection. In embodiments, the non-surgical suprachoroidal injection is achieved by administering the axitinib using an injection device comprising a needle, wherein the needle has an effective length of about 500 to about 2000 microns.

In some embodiments, the method comprises administering axitinib to the eye in at least one dose. In some embodiments, the method comprises administering axitinib to the eye in two doses, wherein the two doses are spaced apart by at least about 1, at least about 2, at least about 3, at least about 4, at least about 5, at least about 6, at least about 7, at least about 8, at least about 9, at least about 10, at least about 11, or at least about 12 months.

In some embodiments, following administration of axitinib to the eye of the subject, the subject experiences no loss in visual acuity as measured by best corrected visual acuity (BCVA). In some embodiments, following administration of axitinib to the eye of the subject, the subject experiences minimal loss in visual acuity as measured by BCVA. In embodiments, the minimal loss in visual acuity is a loss of no more than 2 letters. In further embodiments, the subject experiences an improvement in BCVA of ≥10 letters, ≥15 letters or ≥25 letters, as compared to the subject's visual acuity prior to the administration of the axitinib formulation. In embodiments, the subject experiences no loss in visual acuity as measured by BCVA for 2 months, 3, months, 4 months, 5 months, 6 months, or longer after administration of the axitinib formulation. In embodiments, the subject experiences minimal loss in visual acuity as measured by BCVA for 2 months, 3, months, 4 months, 5 months, 6 months, or longer after administration of the axitinib formulation. In embodiments, the subject experiences an improvement in visual acuity as measured by BCVA for 2 months, 3, months, 4 months, 5 months, 6 months, or longer after administration of the axitinib formulation.

In some embodiments, following administration of axitinib to the eye of the subject, the subject experiences a decrease in retinal thickness from baseline (e.g., retinal thickness such as central subfield thickness (CST) prior to treatment), at any given time point after administration of a drug provided herein, e.g., a decrease of least about 20 μm, or at least about 40 μm, or at least about 50 μm, or at least about 100 μm, or at least about 150 μm or at least about 200 μm, or from about 50-100 μm, and all values in between. In another embodiment, the patient experiences a ≥5%, ≥10%, ≥15%, ≥20%, ≥25% decrease in retinal thickness (e.g., CST) subsequent to administration of axitinib. In one embodiment, change in retinal thickness from baseline is measured as a change in CST, for example, by spectral domain optical coherence tomography (SD-OCT). In embodiments, the subject experiences the decrease in retinal thickness for 2 months, 3, months, 4 months, 5 months, 6 months, or longer after administration of the axitinib formulation. In embodiments, the subject experiences no increase in retinal thickness for 2 months, 3, months, 4 months, 5 months, 6 months, or longer after administration of the axitinib formulation.

In some embodiments, the axitinib is Form IX axitinib. In some embodiments, the axitinib is present in the formulation in an amount of about 0.5 mg/mL to about 100 mg/mL, e.g., about 1 mg/mL to about 10 mg/mL or about 20 mg/mL to about 80 mg/mL. In embodiments, the axitinib is present in the formulation in an amount of about 1 mg/mL or about 40 mg/mL. In some embodiments, the present disclosure provides formulations comprising axitinib and Polysorbate 80. In further embodiments, the present disclosure provides formulations comprising carboxymethylcellulose sodium, Polysorbate 80, sodium chloride, and sodium phosphate. In further embodiments, the formulation comprises Polysorbate 80 at a w/v of about 0.025% to about 0.2%. In some embodiments, the Polysorbate 80 is present in the formulation at an amount of about 0.05% to about 0.1%. In some embodiments, the Polysorbate 80 is present in the formulation at an amount of about 0.05% or about 0.1%. In some embodiments, formulation comprises about 0.04% w/v to about 0.07% w/v sodium phosphate (monobasic monohydrate). In further embodiments, the formulation comprises about 0.059% w/v sodium phosphate (monobasic monohydrate). In some embodiments, the formulation comprises about 0.05% w/v to about 0.09% w/v sodium phosphate (dibasic, anhydrous). In some embodiments, the formulation comprises about 0.079% w/v sodium phosphate (dibasic, anhydrous). In some embodiments, the formulation comprises about 0.5% w/v to about 1.0% w/v sodium chloride. In further embodiments, the formulation comprises about 0.7% w/v to about 0.9% w/v sodium chloride. In some embodiments, the formulation comprises about 0.79% w/v sodium chloride. In some embodiments, the formulation comprises about 0.25% w/v to about 0.75% w/v carboxymethylcellulose sodium. In further embodiments, the formulation comprises about 0.3% w/v to about 0.7% w/v carboxymethylcellulose sodium. In some embodiments, the formulation comprises about 0.5% w/v carboxymethylcellulose sodium. In some embodiments, the axitinib formulation comprises microparticles comprising axitinib. In some embodiments, the microparticles are about 1-20 microns in size for the D₉₀ distribution. In some embodiments, the microparticles are about 1-10 microns in size for the D₅₀ distribution. In some embodiments, the microparticles are about 1-4 microns in size for the D₁₀ distribution. In some embodiments, the microparticles are about 3-5 microns in size for the D₉₀ distribution. In some embodiments, the microparticles are about 3 microns in size for the D₉₀ distribution. In some embodiments, the microparticles are about 10 microns in size for the D₉₀ distribution. In some embodiments, the clinical formulation of CLS-AX comprises axitinib microparticles of about 10 microns in size for the D₉₀ distribution. In some embodiments, the clinical formulation of CLS-AX comprises axitinib microparticles of about 2 microns in size for the D₁₀ distribution. In some embodiments, the clinical formulation of CLS-AX comprises axitinib microparticles of about 5 microns in size for the D₅₀ distribution. In some embodiments, the clinical formulation of CLS-AX comprises axitinib microparticles of about 10 microns for the D₉₀ distribution.

### BRIEF DESCRIPTION OF THE DRAWINGS

FIGS. 1A and 1B show the mean (±SD) CLS-AX concentrations in the indicated tissues following suprachoroidal injection with CLS-AX into the eyes of male Dutch Belted rabbits. FIG. 1A shows the drug remaining (mg) in sclera/choroid/RPE (top panel) and the concentration (in nM) of drug in the retina and vitreous, at the indicated time points up to Day 91 (bottom panel). FIG. 1B shows the concentration of axitinib (ng/g or ng/mL) in the vitreous humor, sclera/chorid/RPE, or retina at the lower (0.03 mg/eye) or higher (0.1 mg/eye) dose.

FIG. 2A shows the fluorescein angiography (FA) grading scale for the laser CNV model in rats. FIG. 2B shows the % of Grade IV lesions at Day 21 in control animals vs. CLS-AX recipient animals. *, Fischer's Exact p-value=0.0002.

FIGS. 3A and 3B show the results of the vascular leakage studies in the CNV model in pigs. FIG. 3A is a bar graph showing the area of vascular leakage 7 days or 14 days after laser treatment to induce retinal lesions. OS=left eye; OD=right eye; BSS=balanced salt solution; SCS=suprachoroidal space. The OS-BSS treatment is shown as the left bar at both time points and the OD-CLS-AX treatment is shown as the right bar at both time points. FIG. 3B provides representative images from BSS-treated eyes and CLS-AX treated eyes.

FIGS. 4A and 4B show the mean (±SEM) isolectin IB4 on retinal flat mount. FIG. 4A provides representative images. Scale bar=100 μm. FIG. 4B shows the mean±SEM Isolectin IB4 signal. Signal in the CLS-AX group was significantly less than that of the BSS treated group (p=0.0297; t-test [27 sites measured OS; 354 sites measured OD]).

### DETAILED DESCRIPTION OF THE INVENTION

This disclosure is generally related to ophthalmic therapies, and more particularly to methods and devices that allow for infusion of a fluid drug formulation into posterior ocular tissues for targeted, localized treatment, for example, for the treatment of diseases and disorders of the eye associated with neovascularization. For example, the diseases and disorders include neovascular age-related macular degeneration (nAMD; also referred to herein as wet AMD), nonexudative AMD, choroidal neovascularization (CNV), retinal vein occlusion (RVO), nAMD associated with RVO, nAMD associated with CNV, retinopathy, diabetic retinopathy, and diabetic macular edema (DME).

In some embodiments, the formulations comprise one or more tyrosine kinase inhibitors and are administered to the suprachoroidal space (SCS) of the eye via a non-surgical means, for example via a hollow microneedle, and/or an injection device comprising a hollow needle wherein the needle has an effective length of about 500 to about 2000 microns. The methods and formulations provided herein allow for effective posterior segment drug delivery, and generally embody the following characteristics: (1) the methods are non-surgical and thus minimally invasive and safe; (2) the drug formulations are administered in such a way that they are well targeted to the posterior segment of the eye and/or the suprachoroidal space (SCS) of the eye and/or the supraciliary space of the eye and/or the supraretinal space of the eye and/or the subretinal space of the eye, while simultaneously limiting drug exposure to the anterior segment or other regions of the eye; (3) the methods and formulations are capable of delivering drug in a sustained and/or controlled manner; (4) the methods and devices are user-friendly. The non-surgical SCS delivery methods and drug formulations for SCS delivery set forth herein achieve these desired characteristics.

Axitinib is a tyrosine kinase inhibitor (TKI) that antagonizes the vascular endothelial growth factor receptors VEGFR-1, VEGFR-2, and VEGFR-3, as well as of the platelet-derived growth factor receptors (PDGFR) and c-Kit receptors. Axitinib was initially approved in 2012 as an oral tablet formulation (INLYTA®) at a dose of 5 mg given twice daily for the treatment of advanced renal cell carcinoma after failure of one prior systemic therapy. An axitinib formulation suitable for delivery to the eye is provided herein. In some embodiments, the axitinib formulation suitable for delivery to the eye provided herein is “CLS-AX.” Exemplary CLS-AX formulations are provided in Table 1A and Table 1B.

The term “suprachoroidal space,” is used interchangeably herein with suprachoroidal, SCS, suprachoroid, suprachoroidia, and the like; and describes the potential space in the region of the eye disposed between the sclera and choroid. This region primarily is composed of closely packed layers of long pigmented processes derived from each of the two adjacent tissues; however, a space can develop in this region as a result of fluid or other material buildup in the suprachoroidal space and the adjacent tissues. Those skilled in the art will appreciate that the suprachoroidal space frequently is expanded by fluid buildup because of some disease state in the eye or as a result of some trauma or surgical intervention. In the present description, however, in some embodiments, the fluid buildup is intentionally created by infusion of a drug formulation into the suprachoroid to create the suprachoroidal space (which is filled with drug formulation). Not wishing to be bound by theory, it is believed that the SCS region serves as a pathway for uveoscleral outflow (i.e., a natural process of the eye moving fluid from one region of the eye to the other through) and becomes a real space in instances of choroidal detachment from the sclera.

As used herein, “non-surgical” ocular drug delivery devices and methods refer to methods and devices for drug delivery that do not require general anesthesia and/or retrobulbar anesthesia (also referred to as a retrobulbar block). Alternatively or additionally, a “non-surgical” ocular drug delivery method is performed with an instrument having a needle with an effective length of less than 2000 microns; and or an instrument having a needle with a diameter of 28 gauge or smaller. Alternatively or additionally, “non-surgical” ocular drug delivery methods do not require a guidance mechanism that is typically required for ocular drug delivery via a shunt or cannula. Non-surgical ocular drug delivery methods provided herein may be used in a clinic or out-patient setting and do not require a hospital setting. As used herein, “surgical” ocular drug delivery includes insertion of devices or administration of drugs by surgical means, for example, via incision to expose and provide access to regions of the eye including the posterior region, and/or via insertion of a stent, shunt, or cannula and/or via a method that requires anesthesia (e.g., general or retrobulbar anesthesia).

The surgical and non-surgical posterior ocular disorder treatment methods and devices described herein are particularly useful for the local delivery of drugs to the posterior region of the eye, for example the retinochoroidal tissue, macula, retinal pigment epithelium (RPE) and optic nerve in the posterior segment of the eye. In one embodiment, the non-surgical methods provided herein can be used to target drug delivery to specific posterior ocular tissues or regions within the eye or in neighboring tissue. In one embodiment, the methods described herein deliver drug specifically to the sclera, the choroid, the Brach's membrane, the retinal pigment epithelium, the subretinal space, the retina, the macula, the optic disk, the optic nerve, the ciliary body, the trabecular meshwork, the aqueous humor, the vitreous humor, and/or other ocular tissue or neighboring tissue in the eye of a human subject in need of treatment. The methods provided herein, in one embodiment, can be used to target drug delivery to specific posterior ocular tissues or regions within the eye or in neighboring tissue.

As provided throughout, in one embodiment, the methods described herein are carried out with a puncture member, which may comprise a hollow or solid microneedle, for example, a rigid microneedle. As used herein, the term “microneedle” refers to a conduit body having a base, a shaft, and a tip end suitable for insertion into the sclera and other ocular tissue and has dimensions suitable for minimally invasive insertion and drug formulation infusion as described herein. Both the “length” and “effective length” of the microneedle encompass the length of the shaft of the microneedle and the bevel height of the microneedle. In some embodiments, the needles used herein are microneedles in that they have an effective length of less than 2000 microns. For example, in some embodiments, the needles useful in the methods described herein are microneedles in that they have an effective length of about 50 microns to about 2000 microns, or about 500 microns to about 1800 microns, or about 700 microns to about 1500 microns, or about 900 microns to about 1200 microns. In some embodiments, the needles useful in the methods described herein are microneedles in that they have an effective length of about 800 microns, about 900 microns, about 1000 microns, about 1100 microns, or about 1200 microns. In some embodiments, the device used to carry out the methods described herein comprises one of the devices disclosed in U.S. Pat. No. 9,539,139, issued Jan. 10, 2017 or International Patent Application Publication No. WO2014/179698 (Application No. PCT/US2014/036590), filed May 2, 2014 and entitled “Apparatus and Method for Ocular Injection,” each of which is incorporated by reference herein in its entirety for all purposes. In some embodiments, the device used to carry out the methods described herein comprises one of the devices disclosed in International Patent Application Publication No. WO2014/036009 (Application No. PCT/US2013/056863), filed Aug. 27, 2013 and entitled “Apparatus and Method for Drug Delivery Using Microneedles,” incorporated by reference herein in its entirety for all purposes. In some embodiments, the microneedle is an SCS microinjector as described herein.

As used herein, the term “hollow” includes a single, straight bore through the center of the microneedle, as well as multiple bores, bores that follow complex paths through the microneedles, multiple entry and exit points from the bore(s), and intersecting or networks of bores. That is, a hollow microneedle has a structure that includes one or more continuous pathways from the base of the microneedle to an exit point (opening) in the shaft and/or tip portion of the microneedle distal to the base.

The microneedle device in one embodiment, comprises a fluid reservoir for containing the therapeutic formulation (e.g., drug or cell formulation), e.g., as a solution or suspension, and the drug reservoir (which can include any therapeutic formulation) being in operable communication with the bore of the microneedle at a location distal to the tip end of the microneedle. The fluid reservoir may be integral with the microneedle, integral with the elongated body, or separate from both the microneedle and elongated body.

In some embodiments, features of the devices, formulations, and methods are provided in U.S. Pat. No. 9,636,332, U.S. Patent Application Publication No. 2018-0042765, International Patent Application Publication Nos. WO2014/074823 (Application No. PCT/US2013/069156), WO2015/195842 (Application No. PCT/US2015/036299), U.S. Patent Publication No. 2019-0269702, WO 2017/120600 (Application No. PCT/US2017/012755), and/or WO2017/120601 (Application No. PCT/US2017/012757), each of which is hereby incorporated by reference in its entirety for all purposes.

In one embodiment, the device used to carry out one of the methods described herein comprises the device described in U.S. Design patent application Ser. No. 29/506,275 entitled, “Medical Injector for Ocular Injection,” filed Oct. 14, 2014, the disclosure of which is incorporated herein by reference in its entirety for all purposes. In one embodiment, the device used to carry out one of the methods described herein comprises the device described in U.S. Patent Publication No. 2015/0051581 or U.S. Patent Publication No. 2017/0095339, which are each incorporated herein by reference in their entireties for all purposes. In some embodiments, such a device is an SCS microinjector as described herein.

As used herein, the terms “about” and “approximately” generally mean plus or minus 10% of the value stated. For example, about 0.5 would include 0.45 and 0.55, about 10 would include 9 to 11, about 1000 would include 900 to 1100.

Further details of possible manufacturing techniques for the microneedles and/or microinjectors provided herein are described, for example, in U.S. Patent Application Publication No. 2006/0086689, U.S. Patent Application Publication No. 2006/0084942, U.S. Patent Application Publication No. 2005/0209565, U.S. Patent Application Publication No. 2002/0082543, U.S. Pat. Nos. 6,334,856, 6,611,707, 6,743,211 and PCT/US2014/36590, filed May 2, 2014, all of which are incorporated herein by reference in their entireties for all purposes.

Any of the methods described herein can be performed use any suitable injector of the types shown and described herein. In some embodiments, in accordance with the methods described herein, the dose of drug has a delivered volume of at least about 20 μL, at least about 50 uL, at least about 100 μL, at least about 200 μL or at least about 500 μL. In one embodiment, the amount of therapeutic formulation delivered into the suprachoroidal space from the devices described herein is from about 10 μL to about 200 μL, e.g., from about 50 μL to about 150 μL. In another embodiment, from about 10 μL to about 500 μL, e.g., from about 50 μL to about 250 μL, is non-surgically administered to the suprachoroidal space. In some embodiments, about 100 μL of a drug formulation is non-surgically administered to the suprachoroidal space. In some embodiments, the drug formulation comprises 100 μL of an axitinib formulation, e.g., CLS-AX.

The SCS drug delivery methods provided herein allow for the delivery of drug formulation over a larger tissue area and to more difficult to target tissue in a single administration as compared to previously known needle devices. Not wishing to be bound by theory, it is believed that upon entering the SCS the drug formulation flows circumferentially from the insertion site toward the retinochoroidal tissue, macula, and optic nerve in the posterior segment of the eye as well as anteriorly toward the uvea and ciliary body. In addition, a portion of the infused drug formulation may remain in the SCS as a depot, or remain in tissue overlying the SCS, for example the sclera, near the microneedle insertion site, serving as additional depot of the drug formulation that subsequently can diffuse into the SCS and into other adjacent posterior tissues.

The term “subject” is used interchangeably herein with the term “patient.” The subject may be any mammal. Preferably, the subject is a human subject. The human subject treated with the methods and devices provided herein may be an adult or a child. In one embodiment, the subject presents with a retinal thickness of greater than 300 μm (e.g., central subfield thickness as measured by optical coherence tomography). In another embodiment, the subject in need of treatment has a BCVA score of ≥20 letters read in each eye (e.g., 20/400 Snellen approximate). In yet another embodiment, the subject in need of treatment has a BCVA score of ≥20 letters read in each eye (e.g., 20/400 Snellen approximate), but 70 letters read in the eye in need of treatment.

Therapeutic response, in one embodiment, is assessed via a visual acuity measurement at one and/or two months post treatment (e.g., by measuring the mean change in best corrected visual acuity (BCVA) from baseline, i.e., prior to treatment). In one embodiment, a patient treated by one or more of the methods provided herein experiences an improvement in BCVA from baseline, at any given time point (e.g., 2 weeks after administration, 4 weeks after administration, 2 months after administration, 3 months after administration), of at least 2 letters, at least 3 letters, at least 5 letters, at least 8 letters, at least 12 letters, at least 13 letters, at least 15 letters, at least 20 letters, and all values in between, as compared to the patient's BCVA prior to administration of axitinib.

In one embodiment, the patient gains about 5 letters or more, about 10 letters or more, 15 letters or more, about 20 letters or more, about 25 letters or more in a BCVA measurement after administration of axitinib, compared to the patient's BCVA measurement prior to undergoing treatment. In even a further embodiment, the patient gains from about 5 to about 30 letters, 10 to about 30 letters, from about 15 letters to about 25 letters or from about 15 letters to about 20 letters in a BCVA measurement, compared to the patient's BCVA measurement prior to treatment with axitinib. In one embodiment, the BCVA gain is about 2 weeks, about 1 month, about 2 months, about 3 months or about 6 months after administration of axitinib. In another embodiment, the BCVA is measured at least about 2 weeks, at least about 1 month, at least about 2 months, at least about 3 months or at least about 6 months after administration of axitinib.

In one embodiment, the BCVA is based on the Early Treatment of Diabetic Retinopathy Study (ETDRS) visual acuity charts and is assessed at a starting distance of 4 meters.

In another embodiment, the patient subjected to a treatment method, e.g., with one of the devices provided herein substantially maintains his or her vision subsequent to the treatment (e.g., a single administration or multiple administrations of axitinib to the suprachoroidal space of the eye), as measured by losing fewer than 15 letters in a best-corrected visual acuity (BCVA) measurement, compared to the patient's BCVA measurement prior to undergoing treatment. In a further embodiment, the patient loses fewer than 10 letters, fewer than 8 letters, fewer than 6 letters, fewer than 5 letters, or fewer than 2 letters in a BCVA measurement, compared to the patient's BCVA measurement prior to undergoing treatment. In some embodiments, the patient experiences no further loss of vision subsequent to treatment with axitinib (e.g., the patient experiences a loss of 0 letter as measured by BCVA). In some embodiments, the patient experiences a gain in vision subsequent to treatment (e.g., a single administration or multiple administrations of axitinib to the suprachoroidal space of the eye). For example, in some embodiments, the patient experiences a gain of at least 2, at least 5, at least 10, at least 15, at least 20, or more letters. In some embodiments, “minimal loss of vision” as used herein means losing no more than 1 letter, no more than 2 letters, no more than 3 letters, no more than 4 letters, no more than 5 letters, no more than 6 letters, no more than 7 letters, no more than 8 letters, no more than 9, letters, no more than 10 letters, no more than 12 letters, or no more than 15 letters. In some embodiments, the patient experiences a minimal loss of vision, relative to the patient's baseline vision prior to treatment, over 2, 6, 12, 18, or 24 months. In some embodiments, the patient experiences a gain in vision, relative to the patient's baseline vision prior to treatment, over 2, 6, 12, 18, or 24 month.

A decrease in retina thickness and/or macula thickness is one measurement of treatment efficacy of the methods provided herein. For example, in one embodiment, patient suffering from nAMD treated by one of the methods provided herein (e.g., administration of a drug (e.g., axitinib) to the suprachoroidal space of an eye) experiences a decrease in retinal thickness from baseline. For example, in one embodiment, the patient experiences a decrease in central subfield thickness (CST) at any given time point after administration of the drug, e.g., a decrease of least about 20 μm, or at least about 40 μm, or at least about 50 μm, or at least about 100 μm, or at least about 150 μm or at least about 200 μm, or from about 50-100 μm, and all values in between. In another embodiment, the patient experiences a ≥5%, ≥10%, ≥15%, ≥20%, ≥25% decrease in retinal thickness (e.g., CST) subsequent to administration of the drug. In some embodiments, the patient experiences a decrease in CST, relative to the patient's baseline CST prior to treatment, for at least 2, 6, 12, 18, or 24 months.

In one embodiment, a patient treated by the methods provided herein experiences a decrease in retinal thickness from baseline (i.e., retinal thickness prior to treatment), at any given time point, of from about 20 μm to about 200 μm, at from about 40 μm to about 200 μm, of from about 50 μm to about 200 μm, of from about 100 μm to about 200 μm, or from about 150 μm to about 200 μm. In one embodiment, change in retinal thickness from baseline is measured as a change in CST, for example, by spectral domain optical coherence tomography (SD-OCT).

In one embodiment, the decrease in retinal thickness is measured about 2 weeks, about 1 month, about 2 months, about 3 months, about 6 months, about 12 months, about 18 months, or about 24 months after administration of the drug. In another embodiment, the decrease in retinal thickness is measured at least about 2 weeks, at least about 1 month, at least about 2 months, at least about 3 months, at least about 6 months, at least about 12 months, at least about 18 months, or at least about 24 months after administration of the drug. In one embodiment, where multiple dosing sessions are employed, a decrease in retinal thickness is sustained by the patient for at least about 2 weeks, at least about 1 month, at least about 2 months, at least about 3 months or at least about 6 months after each drug administration.

A reduction in the frequency and/or severity in ocular lesions within the eye is also one measurement of treatment efficacy of the methods provided herein.

In one embodiment, the suprachoroidal drug dose sufficient to achieve a therapeutic response in a human subject treated with the non-surgical SCS drug delivery method is less than the intravitreal, parenteral, intracameral, topical, or oral drug dose sufficient to elicit the identical or substantially identical therapeutic response. In a further embodiment, the suprachoroidal drug dose is at least 10 percent less than the oral, parenteral or intravitreal dose sufficient to achieve the identical or substantially identical therapeutic response. In a further embodiment, the suprachoroidal dose is about 10 percent to about 25 percent less, or about 10 percent to about 50 percent less than the oral, parenteral, intracameral, topical, or intravitreal dose sufficient to achieve the identical or substantially identical therapeutic response.

In some embodiments, the non-surgical administration of axitinib according to the methods described herein reduces the number and/or frequency of administration of a VEGF modulator to the subject. Thus, in some embodiments, the administration of the axitinib increases the effectiveness and/or durability of the VEGF modulator treatment. For example, in some embodiments, the SCS administration of axitinib results in a need for fewer administrations of a VEGF modulator, and/or results in a longer period of time between administrations of a VEGF modulator.

In some embodiments, the non-surgical administration of axitinib according to the methods described herein results in maintenance of the improved BCVA and/or the improved CST in the subject for at least 12 weeks, at least 16 weeks, at least 20 weeks, at least 24 weeks, at least 30 weeks, at least 36 weeks, at least 40 weeks, at least 44 weeks, at least 48 weeks, or longer after the initial dose of axitinib. In some embodiments, the non-surgical administration of axitinib according to the methods described herein results in maintenance of the improved BCVA and/or the improved CST in the subject for at least 12 weeks, at least 16 weeks, at least 20 weeks, at least 24 weeks, at least 30 weeks, at least 36 weeks, at least 40 weeks, at least 44 weeks, at least 48 weeks, at least 52 weeks, or longer after a second dose of axitinib.

In one embodiment, the non-surgical administration of axitinib to the eye according to the methods provided herein results in a decreased number of deleterious side effects or clinical manifestations in the treated patient as compared to the number of side effects or clinical manifestations caused by the same drug dose administered intravitreally, intracamerally, orally or parenterally; or results in a decreased number of deleterious side effects or clinical manifestations in the treated patient as compared to those caused by administration of a drug previously used to treat the disease.

Examples of side effects and clinical manifestations that can be reduced or ameliorated include, but are not limited to, inflammation, gastrointestinal side effects (e.g., diarrhea, nausea, gastroenteritis, vomiting, gastrointestinal, rectal, and duodenal hemorrhage, hemorrhagic pancreatitis, large intestine perforation black or bloody stools, and/or coughing up blood); hematologic side effects (e.g., leucopenia, anemia, pancytopenia and agranulocytosis, thrombocytopenia, neutropenia, pure red cell aplasia (PRCA), deep venous thrombosis easy bruising, and/or unusual bleeding from the nose, mouth, vagina, or rectum); immunologic side effects/clinical manifestations (e.g., immunosuppression, immunosuppression resulting in sepsis, opportunistic infections (herpes simplex virus, herpes zoster, and invasive candidal infections), and/or increased infection); oncologic side effects/clinical manifestations (e.g., lymphoma, lymphoproliferative disease and/or non-melanoma skin carcinoma); renal side effects/clinical manifestations (e.g. dysuria, urgency, urinary tract infections, hematuria, kidney tubular necrosis, and/or BK virus-associated nephropathy); metabolic side effects/clinical manifestations (e.g. edema, hyperphosphatemia, hypokalemia, hyperglycemia, hyperkalemia. swelling, rapid weight gain, and/or enlarged thyroid); respiratory side effects/clinical manifestations (e.g., respiratory infection, dyspnea, increased cough, primary tuberculosis dry cough, wheezing, and/or stuffy nose); dermatologic side effects/clinical manifestations (e.g., acne, rash, dyshidrotic eczema, papulosquamous psoriatic-like skin eruption rash, blisters, oozing, mouth sores, and/or hair loss); muscoskeletal side effects/clinical manifestations (e.g. myopathy and/or muscle pain), hepatic side effects/clinical manifestations (e.g. hepatoxicity and/or jaundice), abdominal pain, increased incidence of first trimester pregnancy loss, missed menstrual periods, severe headache, confusion, change in mental status, vision loss, seizure (convulsions), increased sensitivity to light, dry eye, red eye, itchy eye, and/or high blood pressure.

As provided throughout, the compositions administered herein in one embodiment, the methods described herein comprise administering a tyrosine kinase inhibitor. Exemplary tyrosine kinase inhibitors for use in the methods described herein include, but are not limited to, Alectinib (Alecensa®); angiokinase inhibitors such as Nintedanib (Vargatev®), Afatinib (Gilotrif®), and Motesanib; Apatinib; Axitinib; Cabozantinib (Cometriq®); Canertinib; Crenolanib; Damnacanthal; Foretinib; Fostamatinib; growth factor receptor inhibitor; Ibrutinib (Imbruvica®); Icotinib; Imatinib (Gleevec®); Linifanib; Mubritinib; Radotinib; T790M; V600E; Vatalanib; Vemurafenib (Zelboraf®); AEE788 (TKI, VEGFR-2, EGFR: Novartis); ZD6474 (TKI, VEGFR-1, -2, -3, EGFR: Zactima: AstraZeneca); AZD2171 (TKI, VEGFR-1, -2: AstraZeneca); SU 11248 (TKI, VEGFR-1, -2, PDGFR: Sunitinib: Pfizer); AG13925 (TKI, VEGFR-1, -2: Pfizer); AG013736 (TKI, VEGFR-1, -2: Pfizer); CEP-7055 (TKI, VEGFR-1, -2, -3: Cephalon); CP-547,632 (TKI, VEGFR-1, -2: Pfizer); GW7S6024 (TKL VEGFR-1, -2, -3: GlaxoSmithKline); GW786034 (TKI, VEGFR-1, -2, -3: GlaxoSmithKline); sorafenib (TKI, Bay 43-9006, VEGFR-1, -2, PDGFR: Bayer/Onyx); SU4312 (TKI, VEGFR-2, PDGFR: Pfizer); AMG706 (TKI, VEGFR-1, -2, -3: Amgen); XL647 (TKI, EGFR, HER2, VEGFR, ErbB4: Exelixis); XL999 (TKI, FGFR, VEGFR, PDGFR, FII-3: Exelixis); PKC412 (TKI, KIT, PDGFR, PKC, FLT3, VEGFR-2: Novartis); AEE788 (TKI, EGFR, VEGFR2, VEGFR-1: Novartis): OSI-030 (TKI, c-kil, VEGFR: OSI Pharmaceuticals); OS1-817 (TKI c-kit, VEGFR: OSI Pharmaceuticals); DMPQ (TKI, ERGF, PDGFR, ErbB2. p56. pkA, pkC); MLN518 (TKI, Flt3, PDGFR, c-KIT (T53518: Millennium Pharmaceuticals); lestaurinib (TKI, FLT3, CEP-701, Cephalon); ZD 1839 (TKI, EGFR: gefitinib, Iressa: AstraZcneca); OSI-774 (TKI, EGFR: Erlotininb: Tarceva: OSI Pharmaceuticals); lapatinib (TKI, ErbB-2, EGFR, and GD-2016: Tykerb: GlaxoSmithKline).

Axitinib is a potent tyrosine kinase inhibitor of vascular endothelial growth factor receptors VEGFR-1, VEGFR-2, and VEGFR-3. These receptors are implicated in pathologic angiogenesis, tumor growth, and metastatic progression of cancer. Axitinib has been shown to potently and selectively inhibit VEGF-mediated signaling and endothelial cell proliferation and survival at picomolar concentrations. Axitinib also inhibits other RTKs at low nanomolar concentrations, including PDGFR-α, PDGFR-β, and c-Kit. The most common metabolites of axitinib, the N-glucuronide (M7) and the sulfoxide (M12) were ≥400-fold less potent against VEGFR-2.

“CLS-AX” is used herein to describe a formulation comprising axitinib. Exemplary formulations are provided in Tables 1A and 1B and throughout the disclosure.

In one embodiment, the tyrosine kinase inhibitor (e.g., axitinib) may be used in combination with one or more agents listed herein or with other agents known in the art, either in a single or multiple formulations. In one embodiment, the agent is a VEGF modulator and is administered intravitreally to the patient in need of treatment. In one embodiment, the VEGF modulator is a VEGF antagonist. In one embodiment, the second drug is a VEGF antagonist including, without limitation, a VEGF-receptor kinase antagonist, an anti-VEGF antibody or fragment thereof, an anti-VEGF receptor antibody, an anti-VEGF aptamer, a small molecule VEGF antagonist, a thiazolidinedione, a quinoline or a designed ankyrin repeat protein (DARPin). In one embodiment, the VEGF antagonist includes, but is not limited to, aflibercept, ziv-aflibercept, bevacizumab, sonepcizumab, VEGF sticky trap, cabozantinib, foretinib, vandetanib, nintedanib, regorafenib, cediranib, ranibizumab, lapatinib, sunitinib, sorafenib, plitidepsin, regorafenib, verteporfin, bucillamine, axitinib, pazopanib, fluocinolone acetonide, nintedanib, AL8326, 2C3 antibody, AT001 antibody, XtendVEGF antibody, HuMax-VEGF antibody, R3 antibody, AT001/r84 antibody, HyBEV, ANG3070, APX003 antibody, APX004 antibody, ponatinib, BDM-E, VGX100 antibody, VGX200, VGX300, COSMIX, DLX903/1008 antibody, ENMD2076, INDUS815C, R84 antibody, KD019, NM3, MGCD265, MG516, MP0260, NT503, anti-DLL4/VEGF bispecific antibody, PAN90806, Palomid 529, BD0801 antibody, XV615, lucitanib, motesanib diphosphate, AAV2-sFLT01, soluble Flt1 receptor, AV-951, Volasertib, CEP11981, KH903, lenvatinib, lenvatinib mesylate, terameprocol, PF00337210, PRS050, SP01, carboxyamidotriazole orotate, hydroxychloroquine, linifanib, ALG1001, AGN150998, MP0112, AMG386, ponatinib, PD173074, AVA101, BMS690514, KH902, golvatinib (E7050), dovitinib, dovitinib lactate (TKI258, CHIR258), ORA101, ORA102, Axitinib (Inlyta, AG013736), PTC299, pegaptanib sodium, troponin, EG3306, vatalanib, Bmab100, GSK2136773, Anti-VEGFR Alterase, Avila, CEP7055, CLT009, ESBA903, GW654652, HMPL010, GEM220, HYB676, JNJ17029259, TAK593, Nova21012, Nova21013, CP564959, smart Anti-VEGF antibody, AG028262, AG13958, CVX241, SU14813, PRS055, PG501, PG545, PTI101, TG100948, ICS283, XL647, enzastaurin hydrochloride, BC194, COT601M06.1, COT604M06.2, MabionVEGF, Apatinib, RAF265 (CHIR-265), Motesanib Diphosphate (AMG-706), Lenvatinib (E7080), TSU-68 (SU6668, Orantinib), Brivanib (BMS-540215), MGCD-265, AEE788 (NVP-AEE788), ENMD-2076, OSI-930, CYC116, Ki8751, Telatinib, KRN 633, SAR131675, Dovitinib (TKI-258) Dilactic Acid, Apatinib, BMS-794833, Brivanib Alaninate (BMS-582664), Golvatinib (E7050), Semaxanib (SU5416), ZM 323881 HCl, Cabozantinib malate (XL184), ZM 306416, AL3818, AL8326, 2C3 antibody, AT001 antibody, HyBEV, bevacizumab (Avastin®), ANG3070, APX003 antibody, APX004 antibody, ponatinib (AP24534), BDM-E, VGX100 antibody (VGX100 CIRCADIAN), VGX200 (c-fos induced growth factor monoclonal antibody), VGX300, COSMIX, DLX903/1008 antibody, ENMD2076, sunitinib malate (Sutent®), INDUS815C, R84 antibody, KD019, NM3, allogenic mesenchymal precursor cells combined with an anti-VEGF antagonist (e.g., anti-VEGF antibody), MGCD265, MG516, VEGF-Receptor kinase inhibitor, MP0260, NT503, anti-DLL4/VEGF bispecific antibody, PAN90806, Palomid 529, BD0801 antibody, XV615, lucitanib (AL3810, E3810), AMG706 (motesanib diphosphate), AAV2-sFLT01, soluble Flt1 receptor, cediranib (Recentin™), AV-951, tivozanib (KRN-951), regorafenib (Stivarga®), volasertib (BI6727), CEP11981, KH903, lenvatinib (E7080), lenvatinib mesylate, terameprocol (EM1421), ranibizumab (Lucentis®), pazopanib hydrochloride (Votrient™), PF00337210, PRS050, SP01 (curcumin), carboxyamidotriazole orotate, hydroxychloroquine, linifanib (ABT869, RG3635), fluocinolone acetonide (Iluvien®), ALG1001, AGN150998, DARPin MP0112, AMG386, ponatinib (AP24534), AVA101, nintedanib (Vargatef™), BMS690514, KH902, golvatinib (E7050), everolimus (Afinitor®), dovitinib lactate (TKI258, CHIR258), ORA101, ORA102, axitinib (Inlyta®, AG013736), plitidepsin (Aplidin®), PTC299, aflibercept (Zaltrap®, Eylea®), pegaptanib sodium (Macugen™, LI900015), verteporfin (Visudyne®), bucillamine (Rimatil, Lamin, Brimani, Lamit, Boomiq), R3 antibody, AT001/r84 antibody, troponin (BLS0597), EG3306, vatalanib (PTK787), Bmab100, GSK2136773, Anti-VEGFR Alterase, Avila, CEP7055, CLT009, ESBA903, HuMax-VEGF antibody, GW654652, HMPL010, GEM220, HYB676, JNJ17029259, TAK593, XtendVEGF antibody, Nova21012, Nova21013, CP564959, Smart Anti-VEGF antibody, AG028262, AG13958, CVX241, SU14813, PRS055, PG501, PG545, PTI101, TG100948, ICS283, XL647, enzastaurin hydrochloride (LY317615), BC194, quinolines, COT601M06.1, COT604M06.2, MabionVEGF, SIR-Spheres coupled to anti-VEGF or VEGF-R antibody, Apatinib (YN968D1), or AL3818.

In embodiments, the compositions and methods provided herein are for use in treating ocular disease and disorders. Exemplary ocular diseases and disorders include, without limitation, wet AMD, nonexudative AMD, CNV, RVO (including central RVO, hemi-RVO, branch RVO), retinopathy, diabetic retinopathy, and diabetic macular edema (DME). In embodiments, the disease or disorder is macular edema (ME). ME may occur in association with and/or due to central RVO, hemiretianl RVO, branch RVO, inflammation, uveitis, or CNV. In embodiments, the disease or disorder is a geographic atrophy, for example, from AMD, degenerative retinal disorders, or hereditary retinal disorders. In embodiments, the disease or disorder is retinal neovascularization. For example, in embodiments, retinal neovascularization can result form ischemic causes such as diabetic retinopathy, central RVO, hemiretinal RVO, branch RVO, central retinal artery occlusion, branch retinal artery occlusion, sickle cell retinopathy, or retinpaty of prematurity. In embodiments, retinal neovascularization can result form inflammatory and uveitic disorders.

Particular conditions in which choroidal neovascularization may occur include wet AMD, angloid streaks, anterior ischemic optic neuropathy, bacterial endocarditis, Best disease, birdshot retinochroidopathy, choroidal hemanioma, chorodial nevi, choroidal nonperfusion, choroidal osteomas, choroidal rupture, choroidermia, chronic retinal deteachment, coloboma of the retina, diabetes mellitus, drusen, endogenous candida endophthalmitis, extrapapillary hematomas of the retinal pigment epithelium, fundus flavimaculatus, an idiopathic condition, macular hole, malignant melanoma, membranoproliferative glomerulonephritis (type II), metallic intraocular foreign body, morning-glory disc syndrome, retinitis pigmentosa, retinochoroidal coloboma, Rubella, sarcoidosis, serpiginous or geographic choroiditis, subretinal fluid drainage, tilted disc syndrome, toxoplasma retinochoroiditis, tuberculosis, Vogt-Koyanagi-Harada syndrome, idiopathic polypoidal choroidal vasculopathy, ocular ischemic syndrome, and carotid stenosis.

### EXAMPLES

The present invention is further illustrated by reference to the following Examples. However, it should be noted that these Examples, like the embodiments described above, are illustrative and are not to be construed as restricting the scope of the invention in any way.

### Example 1. Axitinib Formulation

One exemplary Axitinib formulation, denoted CLS-AX, includes the following components.

TABLE 1A
Exemplary Axitinib Formulation

| | CLS-AX (% w/v |
|---|---|
| Ingredient | or as noted) |
| Axitinib | 40 mg/mL |
| Carboxymethylcellulose Sodium (NaCMC) | 0.5% |
| Polysorbate 80 (PS-80) | 0.1% |
| Sodium chloride | 0.79% |
| Sodium phosphate, monobasic, monohydrate | 0.059% |
| Sodium phosphate, dibasic, anhydrous | 0.079% |
| Water for Injection | q.s. |
| Sodium hydroxide/Hydrochloric acid | Adjust pH to 6.8 |

One exemplary axitinib formulation, also denoted in the table below as CLS-AX, is a 1 mg/mL formulation and includes the following components.

TABLE 1B
Exemplary Axitinib Formulation

| | Composition | Quantity per |
|---|---|---|
| Component | (% w/w) | Batch (5029 g) |
| Axitnib | 0.0994 | 5.00 g + 0.05 g |
| Super Refined Tween | 0.050 | 2.51 g + 0.05 g |
| 80-LQ-(MH), | | |
| Polysorbate 80 | | |
| 7 MF PH Sodium | 0.497 | 30.00 g + 0.30 g |
| Carboxymethylcellulose | | |
| Sodium Chloride, | 0.794 | 47.92 g + 0.48 g |
| Powder | | |
| Sodium Phosphate, | 0.060 | 3.62 g + 0.04 g |
| Monobasic, | | |
| Monohydrate | | |
| Sodium Phosphate, | 0.080 | 4.83 g + 0.05 g |
| Dibasic, Anhydrous, | | |
| Extra Pure | | |
| Water for Injection | 98.4194 | q.s. to |
| (WFI) | | 5,029.0 g + 1% |

In exemplary axitinib formulations, the axitinib is present at a concentration of 1 mg/mL or 10 mg/mL. In addition, in some exemplary axitinib formulations, the polysorbate 80 is present in the formulation at about 0.04% w/v to about 0.05% w/v. Further, in some exemplary embodiments, the sodium chloride is present at a concentration of 0.8%, and/or the sodium phosphate (monobasic, monohydrate) is present at 0.05% w/v; and/or the sodium phosphate (dibasic, anhydrous) is present at 0.085% w/v. Accordingly, the polysorbate 80 concentration may range from about 0.04% w/v to about 0.1% w/v, the sodium chloride concentration may range from about 0.7% w/v to about 0.9% w/v, the sodium phosphate (monobasic, monohydrate) concentration may range from about 0.05% to about 0.06% w/v, and the sodium phosphate (dibasic, anhydrous) may range from about 0.075% w/v to about 0.085% w/v.

In some embodiments, the NaCMC or a similar compound is included in the formulation as a viscosidy modifier. In some embodiments, the polysorbate 80 (PS-80) or a similar agent is included in the formulation as a surfactant wetting agent for the active pharmaceutical ingredient, axitinib. In some embodiments, the sodium chloride is included in the formulation as a tonicity adjuster. In some embodiments, the sodium phosphate, monobasic, monohydrate; and/or the sodium phosphate, dibasic, anhydrous are included as pH buffers. In some embodiments, the sodium hydroxide/hydrochloric acid is included in the formulation as a pH adjuster. In some embodiments, the water for injection is the solvent of the formulation.

In some embodiments, no preservative is present. In some embodiments, the formulation is terminally sterilized via autoclave.

Further information providing the solubility, particle size, viscosity, and other product profile parameters of exemplary CLS-AX compositions (active pharmaceutical ingredient (API); and drug product) are provided in Table 1C.

TABLE 1C
Exemplary Product Profile

| API | Chemical formula | C22H18N4OS | |
|---|---|---|---|
| | Molecular weight | 386.47 | |
| | (g/mol) | | |
| | Melting point (° C.) | 213-215 | |
| | Boiling Point (° C.) | 668.9 ± 55.0 | |
| | Density (g/mL) | 1.4 | |
| | Solubility | DMSO | |
| | | 0.2 μg/mL in water | |
| | Particle size (±2) | D50-2 μm | |
| | | D90-3 μm | |
| Drug | Particle size (±2) | Preclinical | Clinical |
| Product | | D50-1 μm | D10-2 μm |
| | | D90-3 μm | D50-5 μm |
| | | | D90-10 μm |

| | |
|---|---|
| Viscosity (cP) | 6 |

### Example 2. Ocular Distribution of CLS-AX Following Suprachoroidal Administration to Pigmented Rabbits

The purpose of the following studies was to assess the pharmacokinetics and ocular tissue distribution following a single bilateral suprachoroidal microneedle injection of CLS-AX to male pigmented Dutch Belted rabbits. In one study, CLS-AX was administered at a dose of 4 mg/eye (100 μL/injection). In a separate study, CLS-AX was administered at a dose of 0.1 mg/eye or 0.03 mg/eye. The animals' eyes were examined by a board certified veterinary ophthalmologist using a slitlamp biomicroscope and an indirect ophthalmoscope. The exams occurred predose and on the indicated study days prior to sacrifice, as applicable. On specified days, at least two animals/time point were euthanized for the collection of blood (for plasma) and ocular tissues (aqueous humor, vitreous humor, retina, and sclera/choroid-RPE). Plasma and ocular tissues were analyzed for concentrations of CLS-AX using liquid chromatography/mass spectrometry.

For the initial study, the animals were acclimated to study conditions for 16 days prior to dose administration. At dosing, the animals weighed 1721 to 1941 g and were 5 months of age. All animals were housed in individual, suspended, stainless steel cages during acclimation and the test period. Certified Hi-Fiber Rabbit Diet #5325 (PMI) was provided. Water was provided fresh daily, ad libitum. All animals were housed in individual, suspended, stainless steel cages during acclimation and the test period. Environmental controls for the animal room were set to maintain a temperature of 16 to 22° C. (Deviation), a relative humidity of 50±20% (Deviation), and a 12-hour light/12-hour dark cycle. As necessary, the 12-hour dark cycle was interrupted to accommodate study procedures. Each animal was assigned a temporary identification number. At selection, permanent animal numbers were assigned (Deviation). Each animal was uniquely identified with an individually numbered cage card prior to animal selection and an implantable microchip identification device upon assignment to the study. Immediately prior to dosing, animals were anesthetized with an intramuscular (IM) injection of ketamine, dexmedetomidine, and glycopyrrolate. Following application of topical anesthetic, eyes were rinsed with an iodine solution followed by a saline rinse. Animals were not fasted prior to dose administration.

The dosing formulation was drawn up into a 1-mL luer-lock syringe using a standard 21-gauge, 1-inch needle; any bubbles were expressed, and the standard needle was replaced by a 30-gauge microneedle 700 μm in length. A single suprachoroidal injection of 100 μL was given over approximately 5-10 seconds to each eye (3-4 mm from the limbus, in the superior temporal quadrant) by an OSOD representative according to a study-specific procedure. Following the injection, the needle was kept in the eye for approximately 20 seconds before being withdrawn. Upon withdrawal of the microneedle, a cotton-tipped applicator (CTA, dose wipe) was placed over the injection site for approximately 10 seconds; the dose wipe was discarded. The eye was inspected to confirm accuracy of injection by an OSOD representative. The right eye was dosed first; all postdose times were based on the time of dosing of the second (left) eye. Any dosing observations were recorded.

Animals were given an intramuscular (IM) injection of flunixin (2 mg/kg) prior to sedation and approximately 24 hours (0.08 mL per animal) post the first flunixin administration, as applicable. Buprenorphine sustained release (SR, 0.2 mg/kg) was administered subcutaneously (SQ) and bland ophthalmic ointment was applied to each eye upon recovery. Animals were also given neo-poly-bac ointment and atropine ointment topical ocular to both eyes once following dosing on Study Day 1 and twice daily on Study Days 2 and 3.

Twice daily (a.m. and p.m.), animals were observed for mortality and signs of pain and distress. Cageside observations for general health and appearance, with particular attention paid to the eyes, were done once daily.

Body weights were taken on the day of arrival, at the time of animal selection, on the day of dose administration, and weekly throughout the remainder of the study, as applicable.

A board-certified veterinary ophthalmologist conducted ophthalmic examinations predose and on Study Days 4, 15, 28, and 91. At each time point, using a slitlamp biomicroscope to examine the adnexa and anterior portion of each eye, an external examination was conducted. In addition, the eyes were dilated with a mydriatic agent, and the ocular fundus of each eye was examined using an indirect ophthalmoscope.

The following samples were collected for analysis.

Blood and Plasma: Two animals/time point were euthanized with an overdose of sodium pentobarbital and blood (approximately 5 mL) was collected via cardiac puncture into tubes containing K₂EDTA at 24, 72, and 168 hours postdose and on Study Days 15, 29, 61, and 91. Samples were maintained on wet ice until centrifuged to obtain plasma. All plasma samples were placed on dry ice prior to storage at approximately −70° C. until analyzed. The cellular fraction was discarded. Additional blood was collected and discarded to facilitate collection of ocular tissues.

Ocular Tissues: At the time of sacrifice, both eyes were immediately enucleated. The aqueous humor was collected and each eye was flash frozen in liquid nitrogen for 15 to 20 seconds, and subsequently placed on dry ice for at least 2 hours (Deviation). Within approximately one day, the aqueous humor, retina, sclera/choroid (including retinal pigmented epithelium), and vitreous humor tissues were collected. The ocular tissues were rinsed with saline and blotted dry, as appropriate, weighed, and placed on dry ice until stored at approximately −70° C. until analyzed. Residual carcasses and remaining ocular tissues were discarded

Plasma and ocular tissues were analyzed for concentrations of CLS-AX using liquid chromatography/mass spectrometry.

Values from instruments such as balances are reported as generated by (or recorded from) each instrument. Unless otherwise noted, calculated values for mean and standard deviation are reported to three significant figures. Statistical analyses were limited to descriptive statistics such as mean and standard deviation. Because the data were computer-generated and rounded appropriately for inclusion in the report, the use of reported values to calculate subsequent parameters will, in some instances, yield minor variations from those listed in the tables. Dose tables were compiled with values calculated using Excel, Version 14.0 (Microsoft Corporation). As applicable, for individual animals, the maximum concentration (Cₘₐₓ) in plasma, aqueous humor, retina, sclera/choroid-RPE (SCR), and vitreous humor and the time to reach maximum concentration (Tₘₐₓ) were obtained by visual inspection of the raw data. Pharmacokinetic parameters calculated included half-life (t_{1/2}), area under the concentration-time curve from time 0 to the last measurable time point (AUC₀₋ₜ), and area under the concentration-time curve from 0 to infinity (AUC_{0-∞}). Pharmacokinetic parameters were calculated by using Phoenix Winnonlin, version 6.2.1 (Pharsight Corporation).

All animals appeared clinically healthy throughout acclimation and were released from acclimation and approved for use on the study. The suprachoroidal administration of CLS-AX did not have a deleterious effect on body weight over the duration of the study.

Sporadic instances of low food consumption were noted throughout the study. Infrequent occurrences of these observations are considered normal for the species in a laboratory environment.

Results. All animals were free of ophthalmologic findings prior to test article administration. Suprachoroidal administration of CLS-AX at 4 mg/eye (100 μL/injection) was well tolerated through Study Day 91. Subconjunctival white plaques, likely representing test article were commonly observed at later intervals once the initial conjunctival response to the injection had resolved. The observed white plaques were consistent with the dosing observations above, indicating the plaques may be from the minor reflux into the subconjunctival space of CLS-AX following dosing. Clear subconjunctival channels filled with a clear fluid were also observed in some eyes near the injection site on Study Day 15 and thereafter. Although the exact nature of these channels is unclear, they likely represent congested aqueous veins or lymphatic-like vessels. RPE pigment mottling was observed in one eye on Day 91 of the dosing phase, suggesting that this tissue was disturbed by the injection or test article.

White deposits were observed during tissue collections. Up to 61 days postdose, white deposits were observed on the exterior of the eye and could be removed with the bulbar conjunctiva. However, on Study Day 91, attempts were made to dislodge the white deposits from adhering to the sclera once the conjunctiva was removed. These attempts were unsuccessful, suggesting that the deposit was sub-scleral in location. It is possible the appearance of the deposits were more pronounced because the white test article is located between the translucent sclera and the deeply pigmented choroid. These white deposits were not seen upon examination of the fundus, indicating the deposit was most likely CLS-AX located in the suprachoroidal space, which could be observed during external examination of the eyes.

Following many of the injections, small amount of test material may have been trapped under the conjunctiva or within the sclera upon needle withdrawal. The refluxed material may have appeared as subconjunctival white plaques to the examiner. In agreement with the postdose observations and exam findings, white deposits were observed during tissue collections. Up to 61 days postdose, white deposits were observed on the exterior of the eye and could be removed with the bulbar conjunctiva until Study Day 91, suggesting that the deposit was in part suprachoroidal in location.

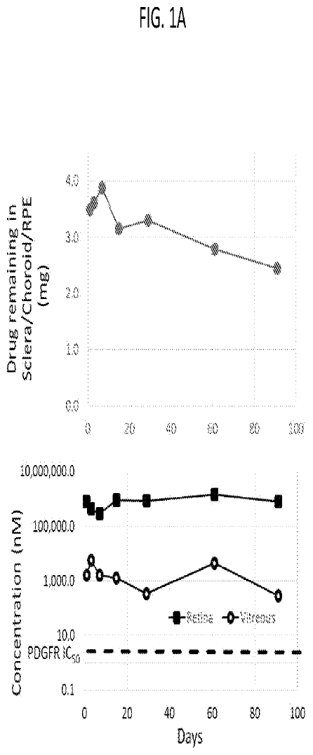

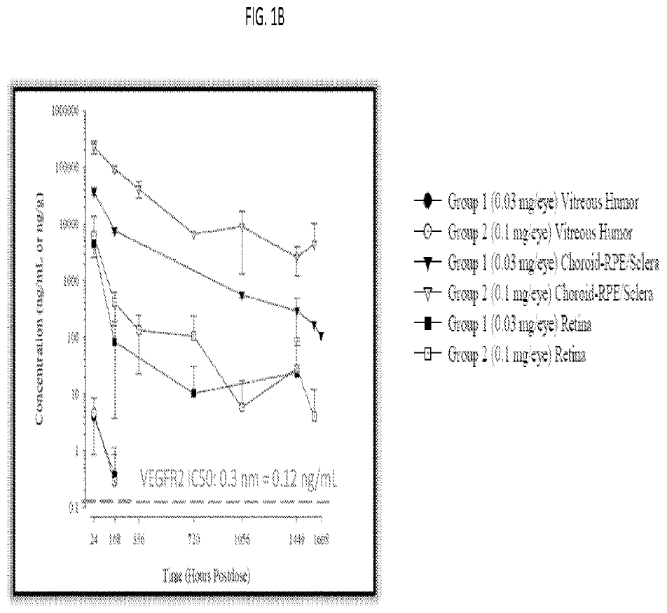

The results of the 4 mg/eye study are provided graphically in FIG. 1A. Pharmacokinetic analysis results are presented in Table 2. The results of the 0.1 mg/eye or 0.03 mg/eye study are provided graphically in FIG. 1B.

Following a single bilateral suprachoroidal administration of CLS-AX (4 mg/eye), the analyte was not observed at quantifiable levels in either plasma or aqueous humor samples. CLS-AX was quantifiable at all time points in vitreous humor, retina, and sclera/choroid-RPE (SCR) after administration of 4 mg/eye. A concentration gradient of CLS-AX in tissues was present, with the dose depot (SCR) the highest, followed by the retina, and finally the vitreous humor with the lowest concentrations (FIG. 1A). The dose depot (SCR) extrapolated initial concentration (C₀) was 17.2 mg/g. The mean concentrations of CLS-AX in the SCR rose slightly on Study Day 4, most likely due to interanimal variability. The dose depot (SCR) concentrations of CLS-AX were the highest early in the study, and then started to decline beyond Study Day 8 through Study Day 91 (FIG. 1A, top panel). The high levels of CLS-AX remaining coincided with observation of sub-scleral white plaques, suggesting that the plaques were remaining dose depot. The elimination half-life (t_{1/2}) was calculated to be 102 days; more than 60% of CLS-AX remained in the SCR at 3 months post injection (FIG. 1A, top panel). The level of CLS-AX in the retina was more than 100,000 fold above the IC50 of axitinib for its receptors (VEGFr and PDGFr) (FIG. 1A, bottom panel). The observed exposure (AUC₀₋ₜ) value of the dose depot was 1260 μg*day/g.

Although CLS-AX is dosed as a suspension, with limited solubility in aqueous solvent, the immediate presence of CLS-AX 24 hours postdose in retina and vitreous humor indicated a burst release of the test article into tissue following dosing. CLS-AX levels in retina and vitreous humor increased over time, to a maximal mean concentration (Cₘₐₓ) of 325 μg/g and 0.857 μg/mL, respectively. The Cₘₐₓ levels were reached in the vitreous humor on Study Day 4 (Tₘₐₓ), and then declined thereafter albeit with some variability. The mean concentrations in retina were similar from Study Day 2 up to Study Day 15, and then increased approximately 3-fold on Study Day 29. The concentrations of CLS-AX in retina were similar to the Cₘₐₓ through Study Day 91. The observed exposure to CLS-AX (AUC₀₋ₜ) in retina and vitreous humor was consistent with the concentration gradient between the two tissues. The retina concentrations of CLS-AX remained above 44 μg/g throughout the duration of the study.

Individual concentrations of CLS-AX in plasma, aqueous humor, retina, sclera/choroid-RPE (SCR), and vitreous humor are presented in Table 2. The concentrations of CLS-AX were determined using liquid chromatography with tandem mass spectrometric (LC-MS/MS) methods. Sample analysis was performed using a verified method. Analyst® software (Version 1.6.2) was used to capture the LC-MS/MS data and integrate the peak areas. Watson LIMS software (Version 7.4.1) was used for data storage, management and reporting.

As noted above, concentration of CLS-AX was below the limit of quantitation (BLQ) in all plasma samples and aqueous humor samples. In retina samples, concentrations of CLS-AX ranged from 2750 ng/mL to 60,100 ng/mL of homogenate and 44,500 ng/g to 1,220,000 ng/g tissue. In SCR, concentrations of CLS-AX ranged from 1,870,000 ng/mL to 3,360,000 ng/mL homogenate and to 9,790,000 ng/g to 24,400,000 ng/g tissue. In the vitreous humor, concentrations of CLS-AX ranged from 60 ng/mL to 6,030 ng/mL.

In summary, following suprachoroidal administration of CLS-AX (4 mg/eye), the analyte was not observed at quantifiable levels (lower limit of quantitation=1 ng/mL) in either plasma or aqueous humor samples throughout the duration of the study. However, CLS-AX was quantifiable at all time points in vitreous humor, retina, and sclera/choroid-RPE (SCR).

In the second study, CLS-AX was detectable in the retina and choroid-RPE/sclera well above the IC50 for the full length of the study (67 days) for both doses (FIG. 1). The concentration detected in the vitreous was significantly lower compared to that in the posterior tissues, and by day 14 CLS-AX was no longer detectable in the vitreous (FIG. 1).

Additional data from the second study are provided below and in Table 1C. Mean axitinib concentrations were maximal on day 1 in SCR, retina, and vitreous humor for both doses. Elimination t_{1/2} values of 257 and 379 hours were calculated for 0.03 and 0.1 mg/eye SCR drug depot, respectively. The 0.1 mg/eye mean Cₘₐₓ and AUC_{0-∞} values were approximately 6- and 7-fold higher than 0.03 mg/eye parameters, respectively.

Following bilateral suprachoroidal administration, axitinib mean retina concentrations reached Cₘₐₓ values of 4480 and 6260 ng/g on study day 2 (tₘₐₓ=24 hours postdose) for 0.03 mg/eye and 0.1 mg/eye, respectively. Beyond Study day 2, the lower dose group mean retina concentrations dropped sharply and were essentially below the limit of quantitation (16.7 ng/g) by day 15, with only 1 of 4 eyes on days 31 and 61 having quantifiable levels of axitinib. The higher dose mean retina concentrations also dropped sharply beyond 24 hours postdose, and were quantifiable in only 1 of 4 eyes on Days 45 through 66. The higher dose mean Cₘₐₓ and AUC₀₋ₜ values were 1.4- and 1.6-fold higher than the lower dose parameters, respectively. These concentration and exposure ratios are less than dose proportional.

Beyond day 2, mean vitreous humor concentrations were quantifiable in only a few eyes in each group on Day 8. The mean Cₘₐₓ values for vitreous humor for the two dose groups were very similar, suggesting that CLS-AX does not readily partition into vitreous humor, independent of dose level.

TABLE 1D
Pharmacokinetic parameters of Axitinib in rabbits dosed via
SCS injection with CLS-AX doses of 0.03 mg/eye or 0.1 mg/eye

| | | | | | | |
|---|---|---|---|---|---|---|
| Dose | | | | AUC₀₋ₜ | AUC_{0-∞} | |
| Administered | C₀ (μg/g | Cₘₐₓ (μg/g | | (μg · day/g | (μg · day/g | |
| (mg/eye) | or mL)^{b} | or mL)^{b} | tₘₐₓ (hour) | or mL) | or mL) | t_{1/2} (hour) |

SCR

| | | | | | | |
|---|---|---|---|---|---|---|
| 0.03 | 48.40 | 37.20 | 24.0 | 8,120.00 | 8,160.00 | 257 |
| 0.1 | 269.00 | 232.00 | 24.0 | 55,600.00 | 57,300.00 | 379 ^{a} |

Retina

| | | | | | | |
|---|---|---|---|---|---|---|
| 0.03 | NC | 4.48 | 24.0 | 414.00 | NC | NC |
| 0.1 | NC | 6.26 | 24.0 | 674.00 | NC | NC |

Vitreous Humor

| | | | | | | |
|---|---|---|---|---|---|---|
| 0.03 | NC | 0.004 | 24.0 | NC | NC | NC |
| 0.1 | NC | 0.0046 | 24.0 | NC | NC | NC |

Taken together, the results of the studies showed that a single bilateral administration of CLS-AX suspension to the suprachoroidal space was well tolerated at all doses tested (4 mg/eye, 0.1 mg/eye, 0.03 mg/eye) and exhibited favorable ocular distribution and pharmacokinetics for durability of treatment of a posterior ocular disorder. Minimal or no systemic exposure, minimal anterior segment exposure, high absorption into the posterior segment, and a much longer half life than expected was achieved. The longer half-life reduces or eliminates the need for repeated injections of axitinib. The problem of effective drug administration to the posterior segment of the eye without detrimental side effects of repeated injections or side effects related to systemic exposure has been a difficult problem for those in the field to solve. With the SCS delivery of an axitinib drug formulation for treatment of a posterior ocular disorder disclosed in the present application and stated in the claims, an unexpected improvement in the method for treatment of a posterior ocular disorder is provided.

TABLE 2
Mean pharmacokinetic parameters in retina, sclera/choroid-RPE, and vitreous humor of
male Dutch Belted rabbits dosed via suprachoroidal injection with Axitinib (4 mg/eye)

| | | Cₘₐₓ | | | AUC₀₋ₜ |
|---|---|---|---|---|---|
| | C₀ | (μg/g | Tₘₐₓ | t_{1/2} | (μg*day/g or |
| Matrix | (μg/g) | or μg/mL) | (days) | (days) | μg*day/mL) |
| Sclera/Choroid-RPE | 17200 | 22000 | NC | 102^{a} | 1260 |
| Retina | NC | 325 | 61 | NC | 23.9 |
| Vitreous Humor | NC | 0.857 | 4 | NC | 0.0314 |

NC Not calculated.
RPE Retinal pigmented epithelium.
^{a}The calculated half-life value should be interpreted with caution as the value was calculated over less than two half-lives.

### Example 3. Rat Laser-Induced Choroidal Neovascularization Model

The purpose of this study was to determine the potential efficacy of suprachoroidal injection of CLS-AX in a laser-induced choroidal neovascularization model with Brown Norway rats using a modified microneedle/SCS microinjector.

On Day 1, prior to the choroidal neovascularization procedure, mydriatic drops were applied to both eyes, and the animals were anesthetized prior to initiation of the following procedures. A 4-spot pattern was made between the major retinal vessels around the optic disc of each eye. Laser parameters were adjusted as required to ensure rupture of Bruch's membrane. Thereafter, groups of male rats (10/group) were administered the control item (sodium chloride for injection, Unites States Pharmacopeia [USP]) or CLS-AX (batch No. 50311-1, 40 mg/mL) under anesthesia into both eyes by freehand bilateral suprachoroidal injection using a 100 μL Hamilton syringe equipped with a modified Clearside microneedle (33 gauge, <400 μm). Dosing was performed on Days 1 and 8 at a dose of 0 or 0.4 mg/eye/dose (0.2 mg/eye per each dose administration), respectively. The target dose volume for each animal was 5 μL/eye/injection.

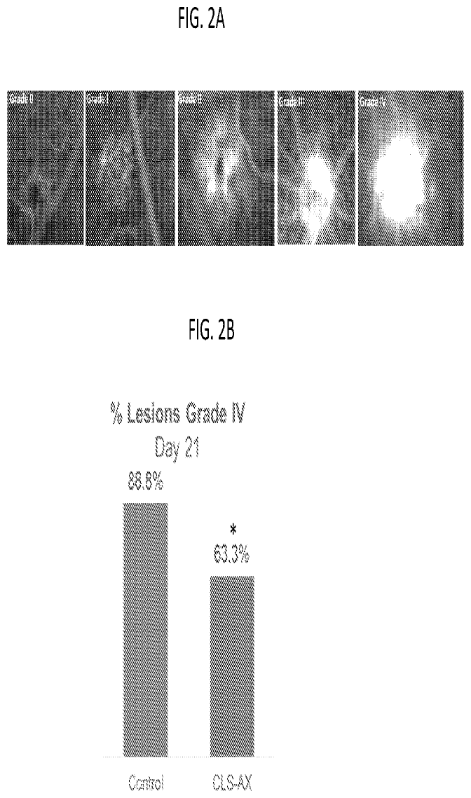

Parameters evaluated during the study included mortality, clinical signs, body weight, food consumption, ophthalmology (indirect ophthalmoscopy and slit-lamp biomicroscopy) once prestudy and on Day 1 post-dose; and fluorescein angiography (FA) once prestudy and on Days 7 (predose), 14, and 21. Fluorescein angiography is a technique used to assess vascular leakage from ocular neovascularization lesions. The individual laser spots on the still images were evaluated for leakage semiquantitatively on a scale of Grade 0 to Grade IV by 2 independent readers, who subsequently determined a consensus score. The scale is shown at FIG. 2A. The animals were sacrificed on Day 22, and the eyes and optic nerves were collected and preserved in Davidson's fixative.

The study design is presented in Table 3.

TABLE 3
Rat Laser-Induced Choroidal Neovascularization Study Design

| | | | Dose | Dose | Number of |
|---|---|---|---|---|---|
| Group | Test | Dose Level | Volume | Concentration | Animals |
| Number | Material | (mg/eye/dose) | (μL) | (mg/mL) | (Male) |
| 1^{a} | Saline for | 0 | 5 | 0 | 10 |
| | Injection USP | | | | |
| 2^{b} | CLS-AX | 0.4 | 5 | 40 | 10 |

Abbreviations:
USP = United States Pharmacopeia;
^{a}Group 1 was dosed on Days 1 and 8.
^{b}Group 2 was dosed on Days 1 and 8 (0.2 mg/eye on each occasion).

There were no deaths, and no treatment-related clinical signs or effects on body weights, body weight gains, or food consumption. Minor retinal iatrogenic trauma resulted from the force applied during the injection procedure, but otherwise no clinically important ocular complications were noted immediately following the first injection.

Administration of CLS-AX using the microneedle resulted in successful suprachoroidal and/or subretinal injections of CLS-AX in most animals, although assessment of the post-injection results in the control group was complicated by the transparency of the control item. CLS-AX appeared white upon ophthalmic examination, and in Group 2, a total of 11/20 eyes had white test item in the SCS as noted from indirect ophthalmoscopy, and 9/20 had subretinal test item. In 4 eyes, the CLS-AX was observed in both subretinal and suprachoroidal spaces. In 5/20 eyes, the test item could be observed near the needle tip and appeared to have been injected subsclerally or suprachoroidally, but in these eyes, possible visualization of the test item upon funduscopy was negative. Exact localization of CLS-AX in these eyes remained uncertain but was unlikely to be subconjunctival as the conjunctiva did not elevate during the injection.

Reflux was minimal in most eyes, and similar in the reference group and CLS-AX group. Six eyes in the reference group and 10 eyes in the CLS-AX group were observed with noted reflux. The higher incidence in the CLS-AX group may have been due to the fact that visualization of the CLS-AX was much easier due to its white color. Reflux observed in this study is likely a result of the difficulty of freehand injection in rats, and is not expected to occur when administering CLS-AX to humans using the microinjector.

Control animals generally showed no decrease in leakage from Days 7 to 21, with the exception of 2 of 20 eyes, from 2 different animals. This rate is typical of the laser-induced choroidal neovascularization model in rats and indicates the model was performing as expected. Decreases in the incidence of clinically important lesions as assessed using FA (scores of 3 or 4) were noted in rats administered CLS-AX, in which 8 of 20 eyes showed improvement (scores of 0 to 2). Comparing the mean scores for control versus treated eyes demonstrated that CLS-AX-treated animals had statistically significant lower scores by Day 21, supporting the activity of CLS-AX in the model. Moreover, the percent of Grade IV lesions at day 21 was significantly less in the CLS-AX group compared to the control group (63.8% and 88.8%, respectively; FIG. 2B). Drug reflux did not appear to correlate with a decreased benefit from CLS-AX treatment.

The study demonstrated that administration of CLS-AX at 0.4 mg/eye/dose once weekly for 2 weeks by suprachoroidal injection was well tolerated in rats and resulted in significant reduction in clinically important lesions by Day 21 compared to control rats treated with saline.

### Example 4. Pig Laser-Induced Choroidal Neovascularization Model

The purpose of this study was to evaluate the effect of a single SC injection of CLS-AX on the development of choroidal neovascularization in a laser-induced porcine model of choroidal neovascularization. This is a model of human ocular posterior segment neovascular diseases occurring in the choroid, such as nAMD.

The potential efficacy of a single SC injection of CLS-AX was assessed in 8 male weanling pigs. On Day 1, mydriatic drops were applied to both eyes and the animals were anesthetized prior to the choroidal neovascularization procedure. The diode laser energy was delivered via a laser indirect headset to create 6 uniform white laser lesions surrounding the optic nerve in each eye. Immediately after laser treatment pigs were administered the control item (balanced salt solution [BSS]) in the left eye and CLS-AX (concentration 40 mg/mL) at a dose of 4 mg in the right eye by freehand SC injection using a syringe equipped with the microneedle (30 gauge, <400 μm) at a dose volume of 100 μL/eye.

Parameters evaluated during the in-life portion of the study included ocular fundus photography and FA immediately following treatment as well as 1 and 2 weeks post treatment. The pigs were euthanized 2 weeks following treatment and the eyes were enucleated and processed for retinal flat mounts. Isolectin IB₄ staining, a technique used to evaluate neovascularization, was performed to quantify neo-formed vessels in retinal flat mounts.

The experimental design is provided in Table 4.

TABLE 4
Pig Laser-Induced Choroidal Neovascularization Study Design

| | N | | | Injection | Sample Collection Times |
|---|---|---|---|---|---|
| Group | (Animals) | Laser (OU) | Drug | Volume | N = 8 animals per time point |
| 1 | 8 | 6 spots around | OS: BSS | 100 μL | Ocular fundus photography (0, 1, |
| | | optic nerve | OD: CLS- | | and 2 weeks after laser) |
| | | | AX 4 mg | | Fluorescein angiography (0, 1, |
| | | | | | and 2 weeks after laser) |
| | | | | | Retinal flat mounts (2 weeks |
| | | | | | after laser [n = 16 eyes]) |

Abbreviations:
BSS = balanced salt solution;
OD = right eye;
OS = left eye;
OU = both eyes

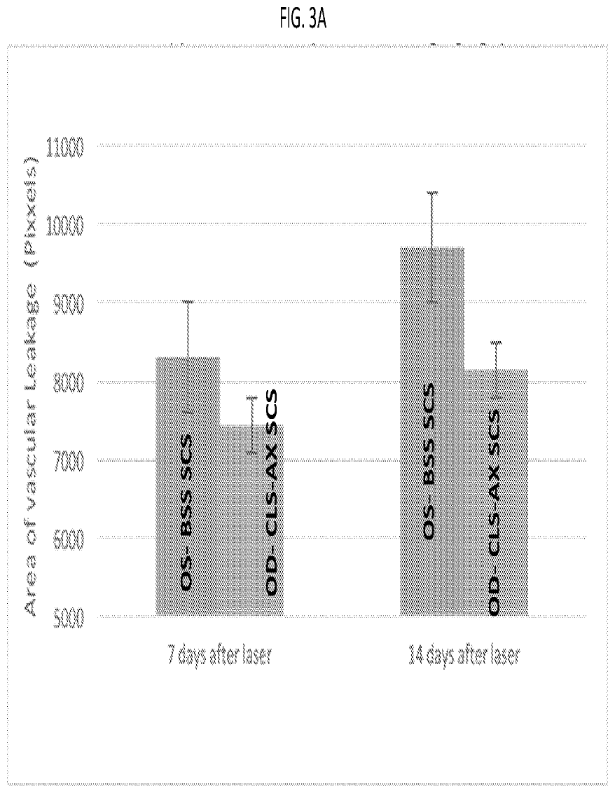

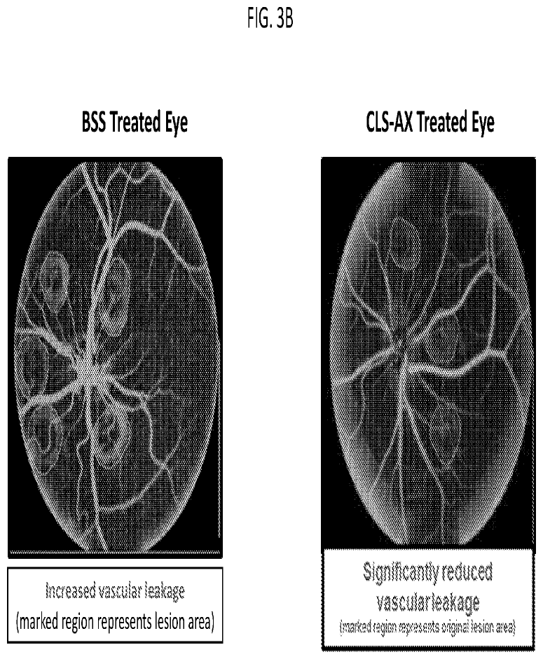

The laser-induced retinal lesions were readily observed by fundus photography and FA. All lesions became less white and defined over time, but there was no visible difference between eyes dosed with BSS (OS [left eye]) or CLS-AX (OD [right eye]) as evaluated immediately after laser treatment and at 1 and 2 weeks after laser treatment. On FA, at 1 and 2 weeks after laser treatment, the eyes treated with CLS-AX had a 10.5% (P=0.009) and 16.0% (P=0.0015) mean reduction in the area of fluorescence, respectively, compared to the control eyes given BSS (FIG. 3A), indicating treatment with CLS-AX significantly reduced vascular leakage and reduced growth of new blood vessels at the site of the retinal laser lesion. Representative images of a BSS treated eye and a CLS-AX treated eye are provided in FIG. 3B.

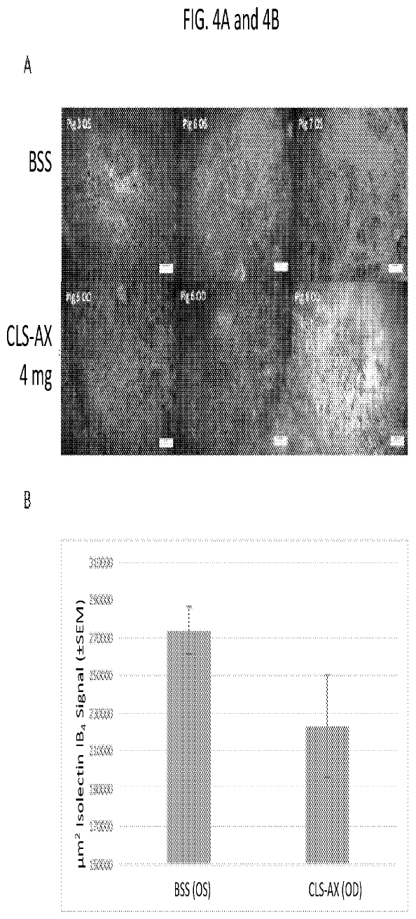

Quantification of neoformed vessels performed on retinal flat mount tissue (measured by isolectin IB₄ signal—a marker of neoformed vasculature) revealed that eyes treated with CLS-AX had reduced isolectin IB₄ signal area by 18% relative to BSS-treated eyes (Representative sample; FIG. 4A; Quantification of mean IB₄ signal, FIG. 4B). These data indicated that CLS-AX treatment significantly suppressed neovascularization relative to the control eyes administered BSS (P=0.0297).

Thus, a single SCS injection of CLS-AX at a dose of 4 mg/eye significantly reduced vascular leakage (as evidenced by reduced fluorescein signal) and growth of new blood vessels (as assessed by reduced isolectin IB₄ signal) at the site of the retinal laser lesion in pigs as compared with the control treatment of BSS.

Taken together, the pharmacokinetic and pharmacodynamic data provided herein shows that CLS-AX is generally well tolerated in rats, rabbits, and pigs upon SCS administration with no overt signs of toxicity. Sustained and high exposure of axitinib was observed in ocular tissues throughout the PK study with highest levels in the sclera/choroid/RPE, followed by the retina, followed by the vitreous. No quantifiable axitinib was detected in either plasma or aqueous humor. In rats, CLS-AX decreased the incidence of clinically important neovascular lesions (scores of 3 or 4) where 8/20 eyes (40%) showed a general improvement with reduction in clinically important lesions by Day 21 compared to control group. In pigs, CLS-AX significantly reduced fluorescein leakage (105% and 16% at week 1) and growth of new blood vessels (18%) at the site of the retinal laser lesion as compared to saline treatment. Accordingly, is a potent anti-angiogenic/anti-neovascularization treatment for nAMD, and his highly effective when non-surgically administered to the SCS of the eye. CLS-AX has potential as a therapy, e.g., a bi-annual therapy, for nAMD, given its pan-VEGF inhibition through receptor blockade as well as the pharmacodynamics effect, prolonged duration, ability to directly target affected tissue layers, and high potency as uncovered in the studies provided herein.

### Example 5. Clinical Study

A Phase 1/2a clinical study will be performed to assess the safety and efficacy of non-surgical administration of axitinib to the suprachoroidal space of the eye of nAMD patients. In part, the doses to be tested are in line with the data obtained in the pharmacodynamics studies disclosed herein.

In developed countries, neovascular age-related macular degeneration (nAMD) is the leading cause of irreversible central blindness (Santarelli, 2015). Although AMD pathogenesis is complex and still not fully understood, many of the mechanisms involved are already partially known and, specifically for the 10-15% of AMD classified as the wet type (nAMD), include the vascular endothelial growth factor (VEGF) signaling pathway. In nAMD, abnormal blood vessel growth, choroidal neovascularization (CNV) in the choriocapillaris, immediately below Bruch's membrane, under the retina and macula leads to leakage of blood, lipids, and serum into the retinal layers and causes the macula to bulge or lift up from its normal position, distorting or destroying central vision. In nAMD, VEGF-A, which acts at VEGF-receptors 1 and 2 (VEGFR-1, VEGFR-2) has been shown to promote abnormal blood vessel growth and is therefore an optimal target for treatment. Currently, anti-VEGF drugs are the standard of care for this condition; however significant unmet need remains for significantly improving and maintaining visual acuity in most patients (Martin, 2012). Furthermore, the current treatment paradigm of frequent intravitreal injections is burdensome; for example, a recent “realworld” retrospective study of nearly 50,000 eyes with wet AMD demonstrated that patients are undertreated with only 7.3 injections on average, yielding only a 1-letter gain at 1 year (Ciulla, 2019).

Primary Endpoint: To assess safety and tolerability of CLS-AX in subjects with neovascular age-related macular degeneration who show stable visual acuity (no loss) following ≥3 injections with an intravitreal anti-VEGF therapy in the preceding 5 months.

Secondary Endpoint: To evaluate and compare the effect of 3 cohort regimens of CLS-AX over 3 months on visual function and anatomy and the need for additional treatment with intravitreal aflibercept.

Study Design: This is a Phase 1/2a open-label, dose-escalation study to assess the safety and tolerability of single doses of CLS-AX administered suprachoroidally following at least 3 prior treatments (the last of which will be administered at the Screening visit) with an intravitreal (IVT) anti-VEGF agent in nAMD subjects. The study design includes 3 dose cohorts of 5 subjects each. Subject eligibility will be established at Visit 1, Screening (Day −28±3 days). Eligible subjects will receive an IVT injection of aflibercept, 2 mg (0.05 mL), at Visit 1, Screening (Day −28±3 days), followed by a suprachoroidal injection of CLS-AX at Visit 2, Baseline (Day 0). Subjects return for safety and tolerability assessments, visual function and ocular anatomy assessments, and the need for additional treatment at Visits 3, 4, and 5 (Weeks 4, 8 and 12), (Follow-up Period). Further, additional treatments will be administered at Visits 3 and 4 (Weeks 4 and 8) based on PRN criteria and will consist of aflibercept (2 mg (0.05 mL) administered by IVT injection unless other therapy is medically necessary. All subjects will be followed until Visit 5 (Week 12, Study Exit) regardless of whether additional therapy is given or not. The 3 dose cohorts will include the following:
Cohort 1 (lowest dose arm): 0.03 mg suprachoroidal injection of CLS-AX
Cohort 2 (mid dose arm): 0.06 mg suprachoroidal injection of CLS-AX
Cohort 3 (high dose arm): 0.10 mg suprachoroidal injection of CLS-AX

All cohorts will be assessed for safety and tolerability and effects on visual function and anatomy as outlined in the time and events schedule.

Number of subjects (planned): Approximately 15.

Inclusion criteria: Subjects are eligible for participation in this study if s/he meets all of the following criteria at the Screening visit (Visit 1) and Baseline visit (Visit 2):

1. Diagnosis of neovascular age-related macular degeneration in the study eye
2. Active subfoveal choroidal neovascularization (CNV) secondary to AMD of any lesion type in the study eye with photos and/or fluorescein angiography (FA) and/or spectral-domain optical coherence tomography (SD-OCD) showing: a. the total lesion area (including blood, neovascularization and scar/atrophy)≤30 mm², b. CNV component area of ≥50% of total lesion area, c. CNV must not be associated with subfoveal hemorrhage, subfoveal fibrosis or subfoveal atrophy.
3. At Screening, two or more anti-VEGF treatments (aflibercept, ranibizumab, bevacizumab, brolucizumab) in the 4 months preceding Screening (Visit 1), with the last treatment administered at least 4 weeks prior to Screening (Visit 1), for neovascular AMD in the study eye with a meaningful response, for example, an improvement in vision and/or exudation, based on the Investigator's opinion.
4. Early Treatment Diabetic Retinopathy Study (ETDRS) best-corrected visual activity (BCVA) score of ≥20 letters read (20/400 Snellen equivalent) and ≤75 letters read (20/32 Snellen equivalent) in the study eye with less than 5 letters change in BCVA between Visit 1 and Visit 2 (Screening and Baseline)
5. Understands the language of the informed consent; willing and able to provide written informed consent prior to any study procedures; willing to comply with the instructions and attend all scheduled study visits
6. At least 50 years of age

Ophthalmic Exclusion criteria: Subjects are ineligible for participation in this study if s/he meets any of the following criteria:

1. Any atrophy or fibrosis in the fovea of the study eye as assessed by spectral-domain optical coherence tomography
2. Has significant media opacity in the study eye precluding evaluation of the retina and vitreous
3. Has macular edema or CNV in the study eye with etiology other than AMD; has any active ocular disease or infection in the study eye other than AMD; has CNV in the study eye with any of the following on photos and/or FA and/or SD-OCT: a. Total lesion area (including CNV, hemorrhage, fibrosis, atrophy)>30 mm2, b. CNV component area of <50% of total lesion area, c. Subfoveal hemorrhage, subfoveal fibrosis or subfoveal atrophy, d. Retinal pigment epithelial tear, Retinal angiomatous proliferation (RAP)
4. Other than IVT injected aflibercept, ranibizumab, bevacizumab, or brolucizumab, prior treatment for CNV in the study eye.
5. Intraocular pressure ≥25 mmHg or uncontrolled glaucoma (open angle or angle closure) in the study eye at Visit 1; subjects are not excluded if intraocular pressure (IOP) is <25 mmHg in the study eye with no more than 1 IOP lowering medications; or cup-to-disc ratio>0.8
6. History of any vitreoretinal surgery (examples include but are not limited to scleral buckle, pars plana vitrectomy, retrieval of a dropped nucleus or intraocular lens) or history of panretinal or macular laser photocoagulation in the study eye; intravitreal injections are acceptable; prior cataract extraction, Yttrium-Aluminum-Garnet laser capsulotomy is allowed, but must not have been within 3 months of Screening (Visit 1)
7. Has had cyclodestructive procedures or filtration surgeries in the study eye in the 3 months prior to Visit 1
8. Has high myopia in the study eye defined as a spherical equivalent>−6 diopters or an axial length≥26 mm. Has a prior history of high myopia, if pseudophakic
9. Has had photocoagulation or cryotherapy in the study eye within the 6 months prior to Visit 1
10. In the study eye, any topical ocular corticosteroid in the 10 days prior to treatment at Visit 2 (Day 0); any intraocular or periocular corticosteroid injection
11. Concomitant therapy with any drug that may be toxic to the lens, retina, or optic nerve
12. At the time of screening, is receiving anti-VEGF therapy in the fellow eye, or is expected to receive anti-VEGF therapy in the fellow eye during the study.

General Exclusion Criteria: Subjects are ineligible for participation in this study if s/he meets any of the following criteria:

12. Female subjects who are pregnant, lactating or planning a pregnancy. Females of childbearing potential must agree to submit to a pregnancy test at screening and agree to use an acceptable method of contraception throughout participation in the study. Acceptable methods of contraception include double barrier methods (condom with spermicide or diaphragm with spermicide), hormonal methods (oral contraceptives, implantable, transdermal, or injectable contraceptives), or an intrauterine contraceptive device with a documented failure rate of less than 1% per year. Abstinence may be considered an acceptable method of contraception at the discretion of the Investigator, but the subject must agree to use one of the acceptable birth control methods if she becomes sexually active.
13. Any uncontrolled systemic disease that, in the opinion of the Investigator, would preclude participation in the study (e.g., unstable medical status including uncontrolled elevated blood pressure, cardiovascular disease, and glycemic control) or put the subject at risk due to study treatment or procedures
14. Has taken systemic corticosteroids at doses greater than 20 mg per day for oral prednisone (or equivalent for other corticosteroids) in the 2 weeks prior to Visit 2; subjects on 20 mg or less per day can be enrolled if no increase in dosing is anticipated for the duration of the study
15. Likely need for hospitalization or surgery within the study period, including planned elective surgery or hospitalization that cannot be deferred
16. Hypersensitivity to any component of the CLS-AX, fluorescein, or to topical anesthetics
17. Currently enrolled in an investigational drug or device study or has used an investigational drug or device within 30 days of Visit 1

Investigational product, dosage and mode of administration: CLS-AX, axitinib injectable suspension, doses at 0.03 mg, 0.06 mg and 0.10 mg in 100 μL into the suprachoroidal space, administered with the SCS Microinjector

Criteria for evaluation include incidence of treatment-emergent adverse events (TEAEs) and serious adverse events, grouped by organ system, relatedness to study treatment, and severity (primary); and incidence/descriptive statistics of changes in ocular safety parameters, number of additional aflibercept treatments following CLS-AX administration, mean change from baseline in central subfield thickness, mean change from baseline in ETDRS BCVA letter score, and the systemic and ocular outcomes (secondary).

All subjects will receive IVT injected aflibercept, 2 mg (0.05 mL), at Screening (Visit 1) followed by a single dose of CLS-AX administered suprachoroidally at Baseline (Visit 2). Safety assessments from the visits at Week 4 (Visit 3), 8 (Visit 4) and 12 (Visit 5) following CLS-AX SC injection will be reviewed. Subjects will be assessed for additional therapy at Visits 3, 4, and 5 (Weeks 4, 8, and 12). Additional therapy will be administered at Visits 3 and 4 (Weeks 4 and 8), consisting of IVT injection of aflibercept 2 mg (0.05 mL) (unless other therapy is medically necessary), based on the “Additional Therapy Criteria” provided below.

The following procedures must be performed before the SC CLS-AX injection (the same day as the injection): assess adverse events, review changes to concomitant medications, perform resting heart rate and blood pressure measurements, collect blood for PK analysis, collect urine for pregnancy test in females of child-bearing potential, and perform ophthalmic assessments on the study eye. Ophthalmic assessments include ETDRS BCVA, slit-lamp biomicroscopy including dilated lens grading, intraocular pressure (IOP) measurement in both eyes, dilated indirect ophthalmoscopy, SD-OCT, and OCT-A. The following photographic evaluations will be performed: fluorescein angiograph and fundus photography.

Eligible subjects will receive suprachoroidal injection of 100 μL of CLS-AX. The following assessments will occur after the suprachoroidal injection: assess adverse events, review changes to concomitant medications, perform resting heart rate (resting 5 minutes) and blood pressure measurements at least 30 minutes after injection, perform 12-lead electrocardiogram at least 30 minutes after injection, collect blood approximately 60 minutes after injection for pharmacokinetic analysis, and perform ophthalmic assessments. Ophthalmic assessments include: indirect ophthalmoscopy immediately after the injection, slit-lamp biomicroscopy, and evaluate IOP 10 to 30 minutes after injection. If IOP remains elevated, the subject must remain on site until the IOP is under control according to the Investigator's best medical judgment. If IOP is <30 mmHg, then the subject may leave the clinic. At non-dosing visit days, the Investigator will contact the subject and assess adverse events and review changes to concomitant medications.

At Visit 3 (Week 4) and Visit 4 (Week 8) and Visit 5 (Week 12), the following procedures are performed: assess adverse events, review changes to concomitant medications, perform resting heart rate and blood pressure measurements, perform 12-lead electrocardiogram, collect blood for PK analysis, collect blood and urine samples for central laboratory tests before fluorescein angiogram, and perform ophthalmic assessments on the study eye. Ophthalmic assessments include ETDRS BCVA, slit-lamp biomicroscopy including dilated lens grading, intraocular pressure (IOP) measurement in both eyes, dilated indirect ophthalmoscopy, SD-OCT, and OCT-A. The following photographic evaluations will be performed: fluorescein angiograph and fundus photography. Subjects will be evaluated for the need of additional therapy with aflibercept.

If the predefined criteria described below (“Additional Therapy Criteria”) are met, then IVT aflibercept will be administered. Following administration, the following assessments will be performed: assess adverse events, review changes to concomitant medications, perform ophthalmic assessments on the study eye. Ophthalmic assessments will include: assess the study eye by indirect ophthalmoscopy immediately after the injection, perform slit-lamp biomicroscopy, and evaluate IOP 10 to 30 minutes after injection. If IOP remains elevated, the subject must remain on site until IOP is under control according to the Investigator's best medical judgment. If IOP is <30 mmHg, then the subject may leave the clinic.

Description of Study Drug: Treatment in each cohort will consist of a single unilateral injection of IVT aflibercept in the study eye at Visit 1 (Screening; Day −14 to −1) and a single unilateral suprachoroidal injection of CLS-AX in 100 μL administered (dosing levels of 0.03 mg, 0.06 mg, or 0.10 mg) on Visit 2 (Day 0). Subjects will be assessed for additional treatment at Visits 3, 4, and 5 (Weeks 4, 8, and 12). Additional treatments will be administered based on PRN criteria below (“Additional Therapy Criteria”) and will consist of IVT aflibercept 2 mg unless other therapy is medically necessary.

Enrollment into the next higher dose cohort will be based on the recommendation of the SMC following review of the safety data. Five subjects will be enrolled into each cohort.

Additional Therapy Criteria: At Visits 3, 4, and 5 (Weeks 4, 8, and 12), subjects will be evaluated for the need for additional therapy for neovascular AMD based on the following criteria. If any of the following criteria are met in the study eye at Visit 3 (Week 4) and Visit 4 (Week 8), then IVT aflibercept (2 mg (0.05 mL) will be administered:
Loss of 10 or more letters in BCVA compared to the best prior study-assessed BCVA in the study eye that is attributed to intra- or sub-retinal fluid observed by the Investigator, or
Increase in central subfield retinal thickness greater than 75 microns from Baseline (Visit 2) in the study eye, or
Presence of vision-threatening hemorrhage due to AMD in the study eye.

Additional treatment will be IVT aflibercept 2 mg unless other therapy is medically necessary; even if additional therapy is given the subjects will still be followed until study exit.

Axitinib systemic blood concentrations will be collected from those subjects who consent, by venipuncture by qualified study personnel, and will be used to estimate standard population PK parameters. A single blood sample will be obtained from each subject at the following time points: Visit 2 (Day 0) pre-dose, Visit 2 (Day 0) approximately 60 minutes post-dose, and Visits 3 and 5 (Weeks 4 and 12).

Best corrected visual acuity (BCVA) will be evaluated by ETDRS using standardized lighting and standardized lanes. The results shall be reported as the number of letters read. Visual acuity testing should precede any examination requiring contact with the eye. In order to provide standardization and well-controlled assessments of BCVA during the study, all BCVA assessments must be performed by trained staff who are certified on the study procedure using certified VA equipment/lanes.

Retinal thickness and disease characterization will be assessed via Spectral Domain Optical Coherence Tomography (SD-OCT). The SD-OCT instrument and technician must be certified before screening any subjects. The technician is encouraged to use the same certified equipment throughout the subject's study participation. All images should be taken by the same technician, whenever possible, on each subject per research site. Images will be sent to the Central Reading Center for analysis and interpretation.

Choroidal neovascular membranes will be classified and follow-up structural changes after the suprachoroidal injection will be assessed via Optical Coherence Tomography Angiography (OCT-A). The OCT-A instrument and technician must be certified before screening any subjects. The technician is encouraged to use the same certified equipment throughout the subject's study participation. All images should be taken by the same technician, whenever possible, on each subject per research site.

Intraocular pressure (IOP) will be measured by applanation tonometry and results will be recorded in mmHg. Where available, Goldmann applanation tonometry should be used at all visits. Tonopens may be used for post-injection pressure checks and in cases where no Goldmann is available. The technician is encouraged to use the same tonometry method throughout the subject's study participation. At baseline, where suprachoroidal CLS-AX is to be administered, IOP will be measured 2 times: before suprachoroidal CLS-AX injection, and after suprachoroidal CLS-AX injection. Tonometers must be calibrated for accuracy before the first subject screening at that site and according to the manufacturer specifications during the study, until the last subject has exited the study at that site.

Slit-lamp biomicroscopy, including magnification, will be performed consistent with standard clinical practice. This procedure should be conducted in the same manner for all subjects and will include an assessment of each of the following as normal or abnormal: eyelids, sclera and conjunctiva, cornea, anterior chamber, iris, and lens. All abnormal findings will be described. Slit lamp examination of the iris is to rule out neovascularization of the iris (NVI).

Cataract Lens Grading: if an abnormal finding of cataract is noted during the slit-lamp examination, the cataract should be graded for nuclear opalescence, cortical opacity, and posterior subcapsular opacity. Graders must verify training on the grading procedures. Cataract classification will be based on the Lens Opacities Classification System III (LOCS III) grading scale (Chylack, 1993).

Indirect ophthalmoscopy should be performed according to the Investigator's standard procedure. This procedure should be the same for all subjects observed at the Investigator's site. The fundus will be examined thoroughly, and the following variables will be assessed as normal or abnormal (including but not limited to): vitreous, retina, choroid, and optic nerve/disc, appearance of vessels, and absence of neovascularization.

Fluorescein angiography will be performed for anatomic assessments and will include the area of fluorescein leakage, area of capillary nonperfusion, the presence of retinal vascular and optic nerve head staining, and retinal pigment epithelium abnormalities. Digital equipment will be registered, and photographers certified for the imaging procedures. De-identified images will be uploaded to the Central Reading Center.

Color fundus photographs will be obtained. It is recommended that the fundus photographs should be taken prior to the fluorescein angiography. All photographs should be taken by the same photographer, whenever possible, on all subjects per research site. Digital equipment will be registered, and photographers certified for the imaging procedures. De-identified images will be uploaded to the Central Reading Center.

Statistical methods: This is an open-label Phase 1 study. The observations and change from baseline will be summarized descriptively for each cohort. Categorical variables will be summarized by counts and percentages and continuous variables by descriptive statistics (n, mean, standard deviation, standard error, median, minimum and maximum). Baseline is defined as the last assessment prior to administration of CLS-AX.

Dose-escalation criteria: All subjects will receive IVT aflibercept at Screening (Visit 1) followed by CLS-AX administered suprachoroidally at Baseline (Visit 2). Safety data will be assessed 4, 8 and 12 weeks post CLS-AX injection. Dose escalation will not proceed to the next highest dose if:
A treatment related serious adverse event is reported
≥2 subjects experience a Grade 3 or higher non-serious treatment relate adverse event, based on the CTCAE severity grading scale
≥3 subjects within the same dose cohort experience a Grade 2 or higher non-serious treatment related adverse event in the same MedDRA system organ class
≥4 subjects within the same dose cohort experience a Grade 2 or higher non-serious treatment related adverse event in any MedDRA system organ class
30 letter decrease in BCVA compared with pre-injection visual activity
Sustained (>15 minutes) loss of light perception due to elevated IOP
20 mmHg increase or decrease in IOP that does not return to pre-injection levels within 7 days of the onset of the event

### REFERENCES

Busbee B G, Ho A C, Brown D M, Heier J S, Suñer I J, Li Z, Rubio R G, Lai P, HARBOR Study Group. Twelve-month efficacy and safety of 0.5 mg or 2.0 mg ranibizumab in patients with subfoveal neovascular age-related macular degeneration. Ophthalmology. 2013; 120:1046-1056.
Cabral T, Lima L H, Mello L G M, Polido J, Correa É P, Akiyoshi Oshima, Jimmy Duong, Pedro Serracarbassa, Caio V. Regatieri, Vinit B. Mahajan, Rubens Belfor. Bevacizumab injection in patients with neovascular age-related macular degeneration increases angiogenic biomarkers. Ophthalmol Retina. 2018; 2(1): 31-37. doi:10.1016/j.oret.2017.04.004.
Chylack L T, Wolfe J K, Singer D M, Leske M C, Bullimore M A, Bailey I L, Friend J, Wu S Y. The lens opacities classification system III. The longitudinal study of cataract study group. Archives of ophthalmology. 1993; 111(6): 831-836.
Ciulla T A, Hussain R M, Pollack J S, Williams D F. Visual Acuity Outcomes and Anti-Vascular Endothelial Growth Factor Therapy Intensity in Neovascular Age-Related Macular Degeneration Patients: A Real-World Analysis of 49,485 Eyes. Ophthalmol Retina. 2019 May 25. pii: S2468-6530(19)30280-5.
Giddabasappa A, Lalwani K, Norberg R, Gukasyan H J, Paterson D, Schachar R A, Rittenhouese K, Klamerus K, Mosyak L, Eswaraka J. Axitinib inhibits retinal and choroidal neovascularization in in-vitro and in-vivo models. Exp Eye Res. 2016, 145: 373-379.
Heier J S, Brown D M, Chong V, Korobelnik J F, Kaiser P K, Nguyen Q D, Kirchhof B, Ho A. Ogura Y, Yancopoulos G D, Stahl N, Vitti R, Berliner A J, Soo Y, Anderesi M, Groetzbach G, Sommerauer B, Sandbrink R, Simader C, Schmidt-Erfurth U. Intravitreal aflibercept (VEGF trap-eye) in wet age-related macular degeneration. Ophthalmology. 2012; 119:2537-2548.
Hu-Lowe D, Zou H Y, Grazzini M L, Hallin M E, Wickman G R, Amundson K, Chen J H, Rewolinski D A, Yamazaki S, Wu E Y, McTigue M A, Murray B W, Kania R S, O'Connor P, Shalinsky D R, Bender S L. Nonclinical antiangiogenesis and antitumor activities of axitinib (AG-13736), an oral, potent, and selective inhibitor of vascular endothelial growth factor receptor tyrosine kinases 1, 2, 3. Clin Cancer Res. 2008; 14(22): 7272-7283.
Kang S, Roh C R, Cho W K, Park K C, Yang K J, Choi H S, So-Hee K, Roh Y J. Antiangiogenic effects of Axitinib, an inhibitor of vascular endothelial growth factor receptor tyrosine kinase, on laser-induced choroidal neovascularization in mice. Curr Eye Res. 2012; 8(1): 119-127.
Lieu C H, Tran H, Jiang Z Q, Mao M, Overman M J, Lin E, Eng C, Morris J, Ellis L, Heymach J V, Kopetz S. The Association of alternate VEGF ligands with resistance to anti-VEGF therapy in metastatic colorectal cancer. PLoS ONE. 2013; 8(10): e77117.
Martin D F, Maureen G M, Fine S L, Ying G, Jaffe G J, Grunwald J E, Toth C, Redford M, Ferris F L. Ranibizumab and Bevacizumab for treatment of neovascular age-related macular degeneration. Ophthalmology. 2012; 119(7):1388-98. doi: 10.1016/j.ophtha.2012.03.053.
Nakano A, Nakahara T, Mori A, Ushikubo H, Sakamoto K, Ishii K. Short-term treatment with VEGF receptor inhibitors induces retinopathy of prematurity-like abnormal vascular growth in neonatal Rats. Exp Eye Res. 2016; 143: 120-131.
Rao P, Lum F, Wood K, Salman C, Burugapalli B, Hall R, Singh S, Parke II D W, Williams G A. Real-world vision in age-related macular degeneration patients treated with single anti-VEGF drug type for 1 year in the IRIS Registry. Ophthalmology. 2018; 125:522e528.
Riquelme M L, Campos-Mollo E, Fernández-Sánchez L. Topical axitinib is a potent inhibitor of corneal neovascularization. Clinical and Experimental Ophthalmology 2018; 46: 1063-1074.
Santarelli M, Diplotti L, Samassa F, Veritti D, Kuppermann B D, Lanzetta P. Advances in pharmacotherapy for wet age-related macular degeneration, Expert Opinion on Pharmacotherapy. 2015; 16(12): 1769-1781, DOI: 10.1517/14656566.2015.1067679.
Schmidt-Erfurth U, Kaiser P K, Korobelnik J F, Brown D M, Chong V, Nguyen Q D, Ho A C, Ogura Y, Simader C, Jaffe G J, Slakter J S, Yancopoulos G D, Stahl N, Vitti R, Berliner A J, Soo Y, Anderesi M, Sowade O, Zeitz O, Norenberg C, Sandbrink R, Heier J S. Intravitreal aflibercept injection for neovascular age-related macular degeneration: ninety-six-week results of the VIEW studies. Ophthalmology. 2014; 121(1):193-201.
Thiele S, Liegl R G, König S, Siedlecki J, Langer J, Eibl K, Haritoglou C, Kampik A, Kernt M. Multikinase Inhibitors as a New Approach in Neovascular Age-Related Macular Degeneration (AMD) Treatment: In Vitro Safety Evaluations of Axitinib, Pazopanib and Sorafenib for Intraocular Use. Klin Monatsbl Augenheilkd 2013; 230: 247-254.
Yuan X, Marcano D C, Shin C S, Hua X, Isenhart L C, Pflugfelder S C, Acharya G. Ocular drug delivery nanowafer with enhanced therapeutic efficacy. ACS Nano. 2015; 9(2):1749-58.

Publications, patents and patent applications cited herein are specifically incorporated by reference in their entireties. While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

1. A method of treating neovascular age-related macular degeneration (nAMD), choroidal neovascularization (CNV), or nAMD associated with CNV in a human subject in need thereof, the method comprising,
non-surgically administering an effective amount of a formulation comprising axitinib to the suprachoroidal space (SCS) of the eye of the human subject in need of treatment, wherein the effective amount comprises between about 0.01 and about 0.3 mg of axitinib.

2. The method of claim 1, wherein the effective amount comprises between about 0.03 mg and about 0.1 mg axitinib.

3. The method of claim 1, wherein the effective amount comprises about 0.03 mg axitinib.

4. The method of claim 1, wherein the effective amount comprises about 0.06 mg axitinib.

5. The method of claim 1, wherein the effective amount comprises about 0.1 mg axitinib.

6. The method of claim 1, wherein the effective amount comprises about 0.3 mg axitinib.

7. The method of any one of claims 1-6, wherein the method comprises administering the axitinib to the subject in a first dose and a second dose, wherein the second dose is administered at least 2, at least 3, at least 4 at least 5, or at least 6 months following the first dose.

8. The method of any one of claims 1-7, wherein the method further comprises administering a VEGF antagonist to the subject.

9. The method of claim 8, wherein the VEGF antagonist is administered via intravitreal injection.

10. The method of claim 8 wherein the VEGF antagonist is selected from the group consisting of aflibercept, bevacizumab, ranibizumab, and pegaptinib sodium.

11. The method of any one of claims 1-10, wherein method results in a decrease in retinal thickness in the treated eye, as measured by Spectral Domain Optical Coherence Tomography (SD-OCT) compared to the patient's retinal thickness in the eye in need of treatment prior to the administration of the formulation.

12. The method of claim 11, wherein the decrease in retinal thickness is ≥25 μm, ≥50 μm, ≥75 μm or ≥100.

13. The method of claim 12, wherein the decrease in retinal thickness is ≥5%, ≥10% or ≥25%.

14. The method of any one of claims 1-10, wherein the method results in a decrease in retinal thickness in the eye, as measured by SD-OCT, compared to the retinal thickness in the eye of a subject who was not administered a formulation comprising axitinib.

15. The method of any one of claims 1-10, wherein the patient does not exhibit an increase in retinal thickness in the eye within 12 weeks after the administration of the formulation comprising axitinib.

16. The method of claim 15, wherein the patient does not exhibit an increase in retinal thickness in the eye within 2, 3, 4, 5, or 6 months after the administration of the formulation comprising axitinib.

17. The method of any one of claims 1-10, wherein the patient experiences minimal loss in vision as measured by best-corrected visual acuity (BCVA) measurement, compared to the patient's BCVA measurement prior to administration of the formulation, wherein minimal loss of vision is losing no more than 2 letters.

18. The method of any one of claims 1-10, wherein the patient experiences minimal loss in vision as measured by best-corrected visual acuity (BCVA) measurement, compared to the patient's BCVA measurement prior to administration of the formulation, wherein minimal loss of vision is losing no more than 5 letters.

19. The method of any one of claims 1-10, wherein the patient experiences no loss in vision as measured by BCVA, compared to the patient's BCVA measurement prior to administration of the formulation.

20. The method of any one of claims 1-10, wherein the patient experiences an improvement in vision, as measured by gaining ≥5 letters, ≥10 letters or ≥15 letters in BCVA measurement, compared to the patient's BCVA prior to administration of the formulation.

21. The method of claim 20, wherein the patient experiences the improvement in BCVA for at least 2, 3, 4, 5, or 6 months after administration of the formulation comprising axitinib.

22. The method of any one of claims 1-10, wherein the patient experiences minimal loss in vision for 2, 3, 4, 5, or 6 months after administration of the formulation comprising axitinib, wherein the minimal loss of vision is losing no more than 2 letters as measured by BCVA.

23. The method of any one of claims 1-10, wherein the patient experiences minimal loss in vision for 2, 3, 4, 5, or 6 months after administration of the formulation comprising axitinib, wherein the minimal loss of vision is losing no more than 5 letters as measured by BCVA.

24. The method of any one of claims 1-10, wherein the patient does not experience a loss of vision within 2, 3, 4, 5, or 6 months after the administration of the formulation comprising axitinib, wherein the vision is measured by BCVA.

25. The method of any one of claims 1-24, wherein the axitinib formulation comprises about 1 mg/mL axitinib.

26. The method of any one of claims 1-25, wherein the axitinib formulation comprise about 0.05% polysorbate 80, about 0.5% sodium carboxymethylcellulose, about 0.79% sodium chloride, about 0.06% sodium phosphate monobasic, and about 0.08% sodium phosphate dibasic.
